(19) United States
(12) Patent Application Publication
(10) Pub. No.: US 2025/0100012 A1
(43) Pub. Date: Mar. 27, 2025

(54) SURFACE STRUCTURE FOR FLUID MANIPULATION

(71) Applicant: Volta Labs, Inc., Boston, MA (US)

(72) Inventors: Udayan UMAPATHI, Somerville, MA (US); William Kai LANGFORD, Cambridge, MA (US); Akim LENNHOFF, Cambridge, MA (US); Devin Laier JUNKINS, Boston, MA (US); Cedric Nicolas Marie VIRY, Cambridge, MA (US)

(21) Appl. No.: 18/973,571

(22) Filed: Dec. 9, 2024

Related U.S. Application Data
(63) Continuation of application No. PCT/US2023/068253, filed on Jun. 9, 2023.
(60) Provisional application No. 63/350,835, filed on Jun. 9, 2022.

(51) Int. Cl. B05D 5/08

(52) U.S. Cl. CPC B05D 5/083

(57) ABSTRACT

The present disclosure describes surface coatings and methods of applying surface coatings to improve the performance of fluid manipulation technologies. The surface coatings may provide increased force applied to the fluid, better fluid position accuracy, improved fluid movement reliability, increased maximum fluid movement speed, resilience against surface fouling, resistance against fluid pinning. Further, the present disclosure describes fluid manipulation apparatuses having surface coatings with improved fluid manipulation performance.

### CROSS-REFERENCE

This application is a continuation of International Application No. PCT/US2023/068253, filed Jun. 9, 2023, which claims the benefit of U.S. Provisional Application No. 63/350,835, filed Jun. 9, 2022, which application is incorporated herein by reference.

### BACKGROUND

A coating is a covering that is applied to the surface of an object, usually referred to as the substrate. The purpose of applying the coating may be decorative, functional, or both.

Functional coatings may be applied to change the surface properties of the substrate, such as adhesion, wettability, corrosion resistance, or wear resistance. In other cases, e.g., semiconductor device fabrication (where the substrate is a wafer), the coating adds a completely new property, such as a magnetic response or electrical conductivity, and forms an essential part of the finished product.

Many industrial coating processes involve the application of a thin film of functional material to a substrate, such as paper, fabric, film, foil, or sheet stock.

Coatings may be applied as liquids, gases or solids e.g., Powder coatings.

### SUMMARY

The present disclosure is generally directed to surface coatings for contacting a fluid. Although specific reference is made to surface coatings for fluid manipulation, such as electrowetting devices, embodiments of the present disclosure may comprise additional uses and applications such as medical devices, implants, laboratory equipment, electromechanical devices, and more.

In one aspect, the present disclosure provides a method of coating a surface for contacting a fluid. The method may include applying a film layer to the surface. In some embodiments, the film layer is non-textured. The method may include applying a liquid layer to the film layer. In some embodiments, the liquid layer has a viscosity of about 0.5 centistokes (cSt) to about 100 cSt. In some embodiments, the liquid layer has a viscosity of about 0 cSt to about 20 cSt. In some embodiments, the liquid layer has a viscosity of about 5 cSt to about 20 cSt. In some embodiments, the liquid layer has a viscosity of about 0.5 cSt to about 20 cSt.

In some embodiments, the liquid layer has an average initial thickness ranging from about 0.01 micrometers (μm) to about 500 μm. In some embodiments, the liquid layer has an average initial thickness ranging from about 10 μm to about 1000 μm.

In some embodiments, the film layer has a Ra of about 100 μm to about 0 μm. In some embodiments, the film layer has a Ra of about 100 nanometers (nm) to about 0 nm. In some embodiments, the film layer has a Ra of about 1000 nanometers (nm) to about 0 nm.

In some embodiments, the liquid layer is a lubricating layer. In some embodiments, lubricating layer is a hydrocarbon layer, a silicone layer, a fluorinated layer, or combinations thereof. In some embodiments, the lubricating layer includes polydimethylsiloxanes, polymethyl hydrogen siloxane/hydrogen silicone oil, amino silicone oil, phenyl methyl silicone oil, Diphenyl silicone oil, vinyl silicone oil, hydroxy silicone oil, cyclosiloxanes, polyalkylene oxide silicones, silicone resins, perfluoropolyether (PFPE), perfluoroalkanes, fluorinated ionic fluid, fluorinated silicone oils, perfluoroalkylether, perfluoro tri-n-butylamine (FC-40), hydrofluoroether (HFE) liquids, ionic liquids, mineral oils, ferrofluids, polyphenyl ether, vegetable oil, esters of saturated fatty and dibasic acids, grease, fatty acids, triglycerides, polyalphaolefin, polyglycol hydrocarbons, other alkanes, or other non-hydrocarbon synthetic oils.

In some embodiments, the liquid layer further includes at least one additive. In some embodiments, the at least one additive is a rheology modifier, filler, solvent, surfactant, dye, or combinations thereof.

In some embodiments, the liquid layer may diffuse into the film layer causing the film layer to swell.

In some embodiments, the liquid layer has a static contact angle with the film layer of about 10 degrees to about 0 degrees. In some embodiments, the liquid layer has a static contact angle with the film layer of about 5 degrees to about 0 degrees.

In some embodiments, the film layer includes one or more polymeric films, inorganic films, composite films, or combinations thereof. In some embodiments, the film layer includes polyethylene, polypropylene, polystyrene, polyetheretherketone (PEEK), polyimide, polyacetal, polysulfone, polyphenylene ether, polyphenylene Sulfide (PPS), polyvinyl chloride, synthetic rubber, natural rubber, neoprene, nylon, polyacrylonitrile, polyvinyl butyral, silicone, parafilm, polyethylene terephthalate, polybutylene terephthalate, polyamides, polyoxymethlyene, polycarbonate, polymethylpentene, polyphenylene oxide (Polyphenyl ether), polyphthalamide (PPA), polylactic acid, synthetic cellulose ethers (e.g., methyl cellulose, ethyl cellulose, propyl cellulose, hydroxyethyl cellulose, hydroxyethyl cellulose, hydroxypropyl cellulose (HPC), hydroxyethyl methyl cellulose, hydroxypropyl methyl cellulose (HPMC), ethyl hydroxyethyl cellulose), paraffins, microcrystalline wax, epoxy, polytetrafluoroethylene (PTFE), tetrafluoroethylene (TFE), fluorinated ethylenepropylene copolymer (FEP), polyvinylidene fluoride (PVDF), perfluoroalkoxytetrafluoroethylene copolymer (PFA), perfluoromethyl vinylether copolymer (MFA), ethylenechlorotrifluoroethylene copolymer (ECTFE), ethylene-tetrafluoroethylene copolymer (ETFE), perfluoropolyether (PFPE), polychlorotetrafluoroethylene (PCTFE), ceramic, borosilicate glass, quartz, alumina, silica, clay minerals, bentonite, kaolinite, vermiculite, graphite, molybdenum disulfide, mica, boron nitride, sodium formate, sodium oleate, sodium palmitate, sodium sulfate, sodium alginate, or any other polymer or ceramic material, or combinations thereof.

In some embodiments, the method further includes modifying the film layer. In some embodiments, modifying includes either surface functionalization or application of a secondary coating. In some embodiments, modifying increases the affinity of the liquid layer for the film layer.

In some embodiments, the film layer has a thickness from about 0.1 μm to about 1000 μm.

In some embodiments, a part of, or all of the surface coating may be removed or replaced. In some embodiments, the surface coating may be permanent.

In some embodiments, the surface coating is applied to a surface intended to contact a fluid. In some embodiments, the surface coating is applied to a surface of a cannula, connector, catheter (e.g., central line, peripherally inserted central catheter (PICC) line, urinary, vascular, peritoneal dialysis, and central venous catheters), catheter connector (e.g., Leur-Lok and needleless connectors), clamp, skin hook, cuff, retractor, shunt, needle, capillary tube, endotracheal tube, ventilator, associated ventilator tubing, drug delivery vehicle, syringe, microscope slide, plate, film, laboratory work surface, well, well plate, Petri dish, tile, jar, flask, beaker, vial, test tube, tubing connector, column, container, cuvette, bottle, drum, vat, tank, organ, organ implant, or organ component (e.g., intrauterine device, defibrillator, corneal, breast, knee replacement, and hip replacement implants), artificial organ or a component thereof (e.g., heart valve, ventricular assist devices, total artificial hearts, cochlear implant, visual prosthetic, and components thereof), dental tool, dental implant (e.g., root form, plate form, and subperiosteal implants), biosensor (e.g., glucose and insulin monitor, blood oxygen sensor, hemoglobin sensor, biological microelectromechanical devices (bioMEMs), sepsis diagnostic sensor, and other protein and enzyme sensors), bioelectrode, endoscope (hysteroscope, cystoscope, amnioscope, laparoscope, gastroscope, mediaslinoscope, bronchoscope, esophagoscope, rhinoscope, arthroscope, proctoscope, colonoscope, nephroscope, angioscope, thoracoscope, esophagoscope, laryngoscope, and encepbaloscope) wound dressing (e.g., bandages, sutures, staples), and combinations thereof.

In another aspect, the present disclosure provides a surface coating for contacting a fluid. The surface coating may include a film layer. In some embodiments, the film layer is non-textured. The surface coating may include a liquid layer. In some embodiments, the liquid layer has a viscosity of about 0.5 centistokes (cSt) to about 100 cSt. In some embodiments, the liquid layer has a viscosity of about 0 cSt to about 20 cSt. In some embodiments, the liquid layer has a viscosity of about 5 cSt to about 20 cSt. In some embodiments, the liquid layer has a viscosity of about 0.5 cSt to about 20 cSt.

In some embodiments, the liquid layer has an average initial thickness ranging from about 0.01 micrometers (μm) to about 500 μm. In some embodiments, the liquid layer has an average initial thickness ranging from about 10 μm to about 1000 μm.

In some embodiments, the film layer has a Ra of about 100 μm to about 0 μm. In some embodiments, the film layer has a Ra of about 100 nanometers (nm) to about 0 nm.

In some embodiment, wherein said liquid layer is a lubricating layer. In some embodiments, the lubricating layer is a hydrocarbon layer, a silicone layer, a fluorinated layer, or combinations thereof. In some embodiments, the lubricating layer includes polydimethylsiloxanes, polymethyl hydrogen siloxane/hydrogen silicone oil, amino silicone oil, phenyl methyl silicone oil, Diphenyl silicone oil, vinyl silicone oil, hydroxy silicone oil, cyclosiloxanes, polyalkylene oxide silicones, silicone resins, perfluoropolyether (PFPE), perfluoroalkanes, fluorinated ionic fluid, fluorinated silicone oils, perfluoroalkylether, perfluoro tri-n-butylamine (FC-40), hydrofluoroether (HFE) liquids, ionic liquids, mineral oils, ferrofluids, polyphenyl ether, vegetable oil, esters of saturated fatty and dibasic acids, grease, fatty acids, triglycerides, polyalphaolefin, polyglycol hydrocarbons, other alkanes, or other non-hydrocarbon synthetic oils.

In some embodiments, the lubricating layer further includes at least one additive. In some embodiments, the at least one additive is a rheology modifier, filler, solvent, surfactant, dye, or combinations thereof.

In some embodiments, the liquid layer may diffuse into said film layer causing the film layer to swell. In some embodiments, the liquid layer has a static contact angle with the film layer of about 10 degrees or less. In some embodiments, the liquid layer has a static contact angle with the film layer of about 5 degrees or less.

In some embodiments, the film layer includes one or more polymeric films inorganic films, composite films, or combinations thereof. In some embodiments, the film layer includes polyethylene, polypropylene, polystyrene, polyetheretherketone (PEEK), polyimide, polyacetal, polysulfone, polyphenylene ether, polyphenylene Sulfide (PPS), polyvinyl chloride, synthetic rubber, natural rubber, neoprene, nylon, polyacrylonitrile, polyvinyl butyral, silicone, parafilm, polyethylene terephthalate, polybutylene terephthalate, polyamides, polyoxymethlyene, polycarbonate, polymethylpentene, polyphenylene oxide (Polyphenyl ether), polyphthalamide (PPA), polylactic acid, synthetic cellulose ethers (e.g., methyl cellulose, ethyl cellulose, propyl cellulose, hydroxyethyl cellulose, hydroxyethyl cellulose, hydroxypropyl cellulose (HPC), hydroxyethyl methyl cellulose, hydroxypropyl methyl cellulose (HPMC), ethyl hydroxyethyl cellulose), paraffins, microcrystalline wax, epoxy, polytetrafluoroethylene (PTFE), tetrafluoroethylene (TFE), fluorinated ethylenepropylene copolymer (FEP), polyvinylidene fluoride (PVDF), perfluoroalkoxytetrafluoroethylene copolymer (PFA), perfluoromethyl vinylether copolymer (MFA), ethylenechlorotrifluoroethylene copolymer (ECTFE), ethylene-tetrafluoroethylene copolymer (ETFE), perfluoropolyether (PFPE), polychlorotetrafluoroethylene (PCTFE), ceramic, borosilicate glass, quartz, alumina, silica, clay minerals, bentonite, kaolinite, vermiculite, graphite, molybdenum disulfide, mica, boron nitride, sodium formate, sodium oleate, sodium palmitate, sodium sulfate, sodium alginate, or any other polymer or ceramic material.

In some embodiments, the film layer is modified. In some embodiments, the modified film layer includes either a functionalized surface or a secondary coating. In some embodiments, the modified film layer has a higher affinity for the liquid layer.

In some embodiments, the film layer has a thickness from about 0.1 μm to 1000 μm.

In some embodiments, a part of, or all of the coating may be removed or replaced. In some embodiments, the coating may be permanent.

In some embodiments, the coating is applied to a surface intended to contact a fluid. In some embodiments, the coating is applied to a surface of a cannula, connector, catheter (e.g., central line, peripherally inserted central catheter (PICC) line, urinary, vascular, peritoneal dialysis, and central venous catheters), catheter connector (e.g., Leur-Lok and needleless connectors), clamp, skin hook, cuff, retractor, shunt, needle, capillary tube, endotracheal tube, ventilator, associated ventilator tubing, drug delivery vehicle, syringe, microscope slide, plate, film, laboratory work surface, well, well plate, Petri dish, tile, jar, flask, beaker, vial, test tube, tubing connector, column, container, cuvette, bottle, drum, vat, tank, organ, organ implant, or organ component (e.g., intrauterine device, defibrillator, corneal, breast, knee replacement, and hip replacement implants), artificial organ or a component thereof (e.g., heart valve, ventricular assist devices, total artificial hearts, cochlear implant, visual prosthetic, and components thereof), dental tool, dental implant (e.g., root form, plate form, and subperiosteal implants), biosensor (e.g., glucose and insulin monitor, blood oxygen sensor, hemoglobin sensor, biological microelectromechanical devices (bioMEMs), sepsis diagnostic sensor, and other protein and enzyme sensors), bioelectrode, endoscope (hysteroscope, cystoscope, amnioscope, laparoscope, gastroscope, mediaslinoscope, bronchoscope, esophagoscope, rhinoscope, arthroscope, proctoscope, colonoscope, nephroscope, angioscope, thoracoscope, esophagoscope, laryngoscope, and encepbaloscope) wound dressing (e.g., bandages, sutures, staples), and combinations thereof.

In another aspect, the present disclosure provides an apparatus for fluid manipulation. In some embodiments, the apparatus includes a substrate comprising one or more electrodes. In some embodiments, the apparatus includes first surface over the substrate. In some embodiments, the first surface includes a surface coating. In some embodiments, the surface coating includes a film layer and a liquid layer.

In some embodiments, the substrate further includes a sealant layer. In some embodiments, the sealant layer includes fluoropolymers, polyurethanes, acrylics, silicones, polyolefins, parylenes, or combinations thereof.

In some embodiments, the first surface is flat, curved, tubular, horizontal, vertical, or any combinations thereof.

In some embodiments, the apparatus further includes a second surface parallel to the first surface.

In some embodiments, the apparatus further includes a gap-filling liquid between the substrate and the first surface. In some embodiments, the gap-filling liquid forms displaced volume between the substrate and the first surface. In some embodiments, the displaced volume has a height between about 0.01 μm to about 500 μm. In some embodiments, the gap-filling liquid is a gel, paste, grease, high viscosity oil, a low viscosity oil, or combinations. In some embodiments, the gap-filling liquid is a silicone paste, lithium grease, silicone grease, thermal paste, or dyed grease.

In some embodiments, the gap-filling liquid is a capillary liquid. In some embodiments, the capillary liquid has a contact angle with the film layer of about 5 degrees to about 0 degrees. In some embodiments, the capillary liquid has a contact angle with the film layer of about 1 degree to about 0 degrees. In some embodiments, the capillary liquid has a contact angle with the substrate or sealant layer of about 5 degrees to about 0 degrees. In some embodiments, the capillary liquid has a contact angle with the substrate or sealant layer of about 1 degree to about 0 degrees.

In some embodiments, the capillary liquid is a hydrocarbon oil, silicone oil, fluorinated oil, or liquid acrylate.

In some embodiments, the gap-filling liquid further comprises at least one additive. In some embodiments, the at least one additive is a rheology modifier, filler, solvent, surfactant, dye, or combinations thereof.

In some embodiments, the gap-filling liquid is either an insulating or conductive liquid.

In some embodiments, the first surface is modified. In some embodiments, the modified first surface comprises either a functionalized surface or a secondary coating. In some embodiments, the modified first surface has a higher affinity for the gap-filling liquid.

In some embodiments, the apparatus further includes a vacuum between the substrate and the first surface.

In some embodiments, the first surface of the apparatus has a working area of about 0.0001 cm² to about 10000 cm².

In some embodiments, the apparatus further includes a frame configured to support the first surface.

In some embodiments, the first surface of the apparatus is configured to contact a fluid to be manipulated. In some embodiments, the apparatus is configured to manipulate the fluid in contact with the first surface. In some embodiments, the manipulated fluid includes inorganic ions, organic ions, proteins, DNA, RNA, surfactants, oil droplets, magnetic beads, nanoparticles, microparticles, polymers, organic compounds, hormones, or combinations thereof. In some embodiments, the manipulated fluid comprises water, ethanol, isopropanol, methanol, acetone, formaldehyde, methyl ethyl ketone, acetamide, ethylene glycol, propylene glycol, dimethyl sulfoxide, dimethylformamide, acetic acid, glycerol, or combinations thereof.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

This specification incorporates herein by reference, in their entireties, each of (A) U.S. patent application Ser. No. 16/287,023, filed on Feb. 27, 2019, (B) International Application No. PCT/US2020/048241, filed on Aug. 27, 2020, and (C) International Application No. PCT/US2022/018549, filed on Mar. 2, 2022.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 is an illustration depicting a fluid on surface experiencing pinning and contact angle hysteresis;

FIG. 2 is an illustration depicting a replaceable surface coating for fluid manipulation, according to some embodiments;

FIGS. 3A-3D are illustrations depicting swelling of the film layer by the liquid layer;

FIG. 4 is an illustration depicting a permanent surface coating for fluid manipulation, according to some embodiments;

FIG. 5A is an illustration depicting various profiles of the first surface and second surface of a fluid manipulation apparatus, according to some embodiments;

FIG. 5B is another illustration depicting various profiles of the first surface and second surface of a fluid manipulation apparatus, according to some embodiments;

FIG. 5C is another illustration depicting various profiles of the first surface and second surface of a fluid manipulation apparatus, according to some embodiments;

FIGS. 6A and 6B are illustrations depicting the relationship between the substrate, the capillary fluid, and the first surface, according to some embodiments;

FIG. 7 is an illustration depicting the construction of a replaceable surface coating for electrowetting, according to some embodiments;

FIG. 8 is an illustration depicting the construction of a permanent surface coating for electrowetting, according to some embodiments;

FIG. 9 is an illustration depicting the construction of a replaceable surface coating for gravitational fluid manipulation, according to some embodiments;

FIG. 10A provides example data depicting maximum droplet velocity as a function of a true viscosity of a liquid layer, according to some embodiments;

FIG. 10B provides example data depicting electrowetting force measured at various electrowetting voltage, according to some embodiments;

FIG. 10C provides example data depicting thickness data for various film regions, according to some embodiments; and

FIG. 10D provides example data depicting roughness values for various film samples, according to some embodiments.

### DETAILED DESCRIPTION

In the following detailed description, reference is made to the accompanying figures, which form a part hereof. In the figures, similar symbols typically identify similar components, unless context dictates otherwise. The illustrative embodiments described in the detailed description, figures, and claims are not meant to be limiting. Other embodiments may be utilized, and other changes may be made, without departing from the scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Although certain embodiments and examples are disclosed below, inventive subject matter extends beyond the specifically disclosed embodiments to other alternative embodiments and/or uses, and to modifications and equivalents thereof. Thus, the scope of the claims appended hereto is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments, however, the order of description should not be construed to imply that these operations are order dependent. Additionally, the structures, systems, and/or devices described herein may be embodied as integrated components or as separate components.

For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

The present disclosure describes surface coatings and methods of applying surface coatings to improve the performance of fluid manipulation technologies. The surface coatings of the present disclosure may provide increased force applied to the fluid, better fluid position accuracy, improved fluid movement reliability, increased maximum fluid movement speed, resilience against surface fouling, resistance against fluid pinning, faster heat transfer.

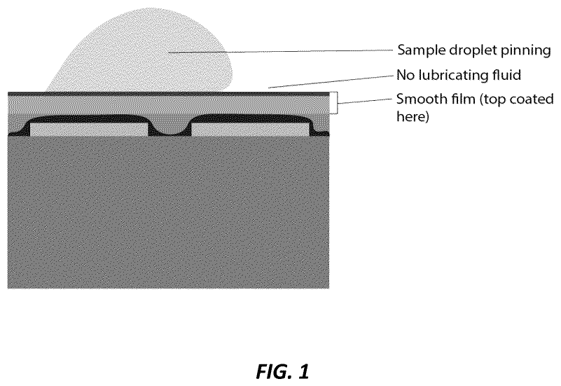

The chemistry and texture of the surface coating govern the ease of fluid manipulation. As a result of the chemical makeup and physical texture, a fluid on a surface may experience two phenomena when manipulated: pinning and contact angle hysteresis. Pinning occurs when a fluid or its constituents, for example DNA or magnetic beads, stick to surface defects as it is being manipulated. Contact angle hysteresis is the difference in the advancing and the receding contact angles for a fluid in motion. In an electrowetting device, droplet pinning and a high contact angle hysteresis, reduce efficiency since higher voltages are required for fluid manipulation and satellite droplets may be left behind. FIG. 1 is an illustration depicting a fluid on a surface experiencing pinning and contact angle hysteresis.

One method to reduce pinning and contact angle hysteresis involves applying a lubricating liquid over a textured surface or porous surface, where the surface texturing helps to support the lubricating liquid. However, textured surfaces can be expensive and time consuming to manufacture which prevents their use in large volume consumables. If used as a reusable surface, textured surfaces may lead to sample-to-sample contamination. Quality control of textured surfaces having nano- or micropatterned surfaces may also be challenging. Surfaces features can easily be damaged after manufacturing. Damage to these textured surfaces can cause pinning and fouling. Non-damaged textured surfaces may also exhibit fouling, undesired droplet pinning and contact angle hysteresis. All of these phenomena can negatively impact fluid manipulation performance.

The surface coatings of the present disclosure are smooth and non-porous and allow for low-cost, high performance, durable, replaceable or permanent surfaces, and may be used with fluid manipulation technologies such as electrowetting for a variety of applications including biological sample processing and chemical synthesis. The surface coatings include a non-textured or smooth film layer and a liquid layer acting as a lubricant to reduce pinning and contact angle hysteresis. Because the film layer is non-textured, the liquid layer must be adequately viscous to prevent the liquid layer from being displaced by the manipulated fluid while minimizing drag forces.

### Definitions

Unless defined otherwise, all terms of art, notations, and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range of formats. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, a description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including combinations thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative, or quantitative and qualitative determinations. Assessing can be relative or absolute. “Detecting the presence of” can include determining the amount of something present in addition to determining whether it is present or absent depending on the context.

The term “about” or “approximately” as used herein when referring to a measurable value such as an amount or concentration and the like, is meant to encompass variations of 20%, 10%, 5%, 1%, 0.5%, or even 0.1% of the specified amount. For example, “about” can mean plus or minus 10%, per the practice in the art. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, up to 5-fold, or up to 2-fold, of a value. Where particular values can be described in the application and claims, unless otherwise stated the term “about” may be assumed to encompass the acceptable error range for the particular value. Also, where ranges, subranges, or both, of values, can be provided, the ranges or subranges can include the endpoints of the ranges or subranges.

Where values are described as ranges, it may be understood that such disclosure includes the disclosure of all possible sub-ranges within such ranges, as well as specific numerical values that fall within such ranges irrespective of whether a specific numerical value or specific sub-range is expressly stated.

The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has,” or “includes” one or more steps is not limited to possessing only those one or more steps and also covers other unlisted steps.

The term “droplet”, as used herein, generally refers to a discrete or finite volume of a fluid (e.g., a liquid). A droplet may be generated by one phase separated from another phase by an interface. The droplet may be a first phase phase-separated from another phase. The droplet me include a single phase or multiple phases (e.g., an aqueous phase containing a polymer or an emulsion). The droplet may be a liquid phase disposed adjacent to a surface and in contact with a separate phase (e.g., gas phase, such as air).

The term “biological sample,” as used herein, generally refers to a biological material. Such biological material may display bioactivity or be bioactive. Such biological material may be, or may include, a deoxyribonucleic acid (DNA) molecule, a ribonucleic acid (R A) molecule, a polypeptide (e.g., protein), or any combination thereof. A biological sample (or sample) may be a tissue sample, such as a biopsy, core biopsy, needle aspirate, or fine needle aspirate. The sample may be a fluid sample, such as a blood sample, urine sample, stool sample, or saliva sample. The sample may be a skin sample. The sample may be a cheek swab. The sample may be a plasma or serum sample. The sample may be a plant derived sample, water sample or soil sample. The sample may be extraterrestrial. The extraterrestrial sample may contain biological material. The sample may be a cell-free (or cell free) sample. A cell-free sample may include extracellular polynucleotides. Extracellular polynucleotides may be isolated from a bodily sample that may be selected from a group consisting of blood, plasma, serum, urine, saliva, mucosal excretions, sputum, stool and tears. The sample may include a eukaryotic cell or a plurality thereof. The sample may include a prokaryotic cell or a plurality thereof. The sample may include a virus. The sample may include a compound derived from an organism. The sample may be from a plant. The sample may be from an animal. The sample may be from an animal suspected of having or carrying a disease. The sample may be from a mammal.

The term “electrowetting,” as used herein, generally refers to any liquid handling technology which uses voltage applied to electrodes or other conductors to move fluids on a surface. The surface tension and wetting properties of a fluid may be altered by electric fields using the electrowetting effect. The electrowetting effect may arise from the change in solid-liquid contact angle due to an applied potential difference between the solid and the liquid. When the fluid is provided as a droplet, differences in wetting surface tension may vary over the width of the droplet, and corresponding change in contact angle, may provide motive force to cause the droplet to move, without moving parts or physical contact.

The term “smooth surface” as used herein, generally refers to non-porous surfaces which are not patterned to improve hydrophobic properties of the surface. A fluid manipulating surface is smooth if there is at least one 100 μm² portion of the surface which is meant to come in contact with a manipulated fluid where any 20 μm² portion has a roughness average (“Ra”) less than 10 μm and a Wenzel roughness factor below 2, wherein the Wenzel roughness factor is defined as the ratio of the real surface area of a surface to the projected surface area of a surface. Folds, wrinkles, and other surface defects do not prevent a film from being considered smooth by this definition.

The section headings used herein are for organizational purposes only and are not to be construed as limiting the subject matter described.

### Surface Coatings for Contacting a Fluid

In one aspect, the present disclosure provides a surface coating for contacting a fluid. In some embodiments, contacting a fluid comprises manipulating a fluid. In some embodiments, manipulating a fluid comprises performing one or more operations, such as moving the fluid on the surface.

In some embodiments, the surface coating is multilayered. In some embodiments, the surface coating has two layers. In some embodiments, the surface coating has three layers. In some embodiments, the surface coating has four layers. In some embodiments, the surface coating has five layers. In some embodiments, the surface coating has six layers. In some embodiments, the surface coating has seven layers. In some embodiments, the surface coating has eight layers. In some embodiments, the surface coating has nine layers. In some embodiments, each layer may serve a different purpose and improve fluid handling performance, such as electrowetting performance, in multiple ways.

In some embodiments, the layers may comprise surface modifications secondary coatings, films, adhesives, fluids, vacuum, or combinations thereof.

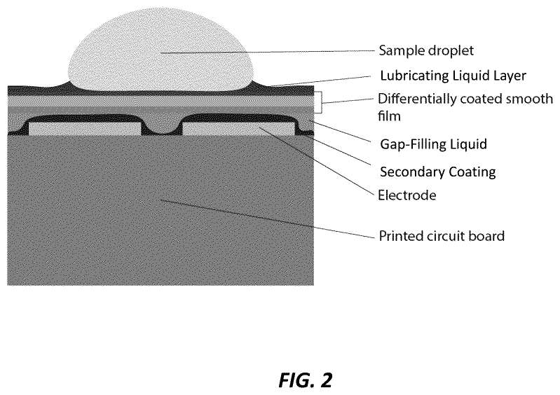

In some embodiments, the surface coating comprises a film layer and a liquid layer. FIG. 2 illustrates a replaceable surface coating for fluid manipulation. As illustrated in FIG. 2, a replaceable surface coating may include a differentially coated smooth film layer and a lubricating liquid layer.

In some embodiments, the film layer has a thickness from about 0.1 micrometers (“μm”) to about 1000 μm. In some embodiments, the film layer has a thickness of at least about 0.1 μm, 0.2 μm, 0.3 μm, 0.4 μm, 0.5 μm, 0.6 μm, 0.7 μm, 0.8 μm, 0.9 μm, 1 μm, 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1000 μm, or any values therebetween. In some embodiments, the film layer is at most about 1000 μm, 900 μm, 800 μm, 700 μm, 600 μm, 500 μm, 400 μm, 300 μm, 200 μm, 100 μm, 90 μm, 80 μm, 70 μm, 60 μm, 50 μm, 40 μm, 30 μm, 20 μm, 10 μm, 9 μm, 8 μm, 7 μm, 6 μm, 5 μm, 4 μm, 3 μm, 2 μm, 1 μm, 0.9 μm, 0.8 μm, 0.7 μm, 0.6 μm, 0.5 μm, 0.4 μm, 0.3 μm, 0.2 μm, 0.1 μm or any values therebetween.

Ra, or arithmetic mean height, may be a measure of surface roughness. Ra may be determined by averaging the absolute values of profile heigh deviations from a mean line, recorded over an evaluation area. In some embodiments, Ra is the arithmetic average of the absolute values of the profile height deviations from the mean line of a particular surface, as measured by a profilometer. The Ra value may be expressed in, e.g., μm. The higher the Ra value, the rougher the surface. For example, a surface with an Ra value of 0.1 μm, may, in some cases, be considered to be smooth, while a surface with an Ra value of 1 μm may, in some cases, be considered to be rough. Other measures of surface roughness that may, in some embodiments, be suitable are root mean square (e.g., calculated by taking the square root of the arithmetic mean of the square profile heigh deviations from the mean line), mean line averaging (e.g., calculated by averaging the absolute values of profile heigh deviations from a mean line, without regard to sign), or total heigh (e.g., calculated as the difference between the highest peak and the deepest valley over an evaluation length).

In some embodiments, the film layer may be non-textured. In some embodiments, non-textured may refer to a surface that is completely smooth (e.g., has no variation in height of profile). In some embodiments, non-textured may refer to a surface that has small deviation in height of profile or deviation within an acceptable or predetermined range. For example, the Ra of the non-textured film layer may be about 100 nm to about 0 nm. In some embodiments, the Ra of the non-textured film layer may be about 100 μm to about 0 μm. In some embodiments, the non-textured film layer has an Ra of least about 0 μm, 0.01 μm, 0.02 μm, 0.03 μm, 0.04 μm, 0.05 μm, 0.06 μm, 0.07 μm, 0.08 μm, 0.09 μm, 0.1 μm, 0.2 μm, 0.3 μm, 0.4 μm, 0.5 μm, 0.6 μm, 0.7 μm, 0.8 μm, 0.9 μm, 1 μm, 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, or any values therebetween. In some embodiments, the non-textured film layer has an Ra of at most about μm, 90 μm, 80 μm, 70 μm, 60 μm, 50 μm, 40 μm, 30 μm, 20 μm, 10 μm, 9 μm, 8 μm, 7 μm, 6 μm, 5 μm, 4 μm, 3 μm, 2 μm, 1 μm, 0.9 μm, 0.8 μm, 0.7 μm, 0.6 μm, 0.5 μm, 0.4 μm, 0.3 μm, 0.2 μm, 0.1 μm, 0.09 μm, 0.08 μm, 0.07 μm, 0.06 μm, 0.05 μm, 0.04 μm, 0.03 μm, 0.02 μm, 0.01 μm, 0 μm, or any values therebetween.

In some embodiments, the instant disclosure comprises a low surface energy oil formed via slippery liquid coating and liquid-on-liquid electrowetting (LLEW). The thin film of oil may be formed on a low surface energy textured or un-textured solid surface. The solid (e.g. a dielectric film) and the lubricating oil may be selected such that the lubricating oil preferentially wets the solid. In some embodiments, the solid (e.g. a dielectric film) and the lubricating oil may be selected such that the lubricating oil wets the solid entirely, and remains non-interacting with the liquid (e.g. a droplet or bodily fluids if the surface is used to coat an implant). Once the bulk of the solid is covered with oil, additional oil may be added above the solid. The self-leveling nature of the oil layer on the top may hide any non-uniformity in the topography of the underlying surface. Thus, a surface of an electrode array with very high roughness (tens of micrometers) may be translated to a nearly-molecular-level smooth surface with a thin layer of lubricating oil. This molecular-level smooth surface may offer very little friction to droplet motion, and droplets may experience little to no droplet pinning. Droplets on such a smooth surface may have very small contact angle hysteresis (as low as) 2°. The resulting low contact angle hysteresis and absence of droplet pinning may lead to very low actuation voltage (from 1V to 100V) with robust droplet manipulation. Oil on top of the solid may be trapped on top of the solid by the wetting interaction between the oil and the solid. The oil layer may have sufficient affinity for and molecular interaction with the solid's surface to reduce the influence of gravity. Since the oil does may not leave the surface of the solid, the droplet or bodily fluids interacting with the solid may ride on the lubricating oil and may interact with the surface of the lubricating oil and not with the underlying solid. As a result, the droplet or bodily fluids may leave little to no trail on the underlying solid. If the oil is immiscible with the droplet or bodily fluids, the droplet or bodily fluids may move on the liquid layer without any contamination between two consecutive droplets crossing paths or different bodily fluids. The lubricating oil may be any low-energy oil such as silicone oil, DuPont Krytox oil, Fluorinert FC-70 or other oil. The lubricating oil may be selected such that the oil is immiscible with liquid droplets or bodily fluids. A lubricant that is immiscible with the droplet solvent may improve the ability of the droplet to ride over the lubricant or oil with less diffusion of contents from the droplet into the oil and vice-versa. The viscosity of the lubricating oil may affect droplet mobility during electrowetting; with lower viscosity promoting higher mobility. Suitable lubricating oils may be non-volatile and immiscible with the riding droplet of interest. If the droplet contains biological constructs, a biocompatible oil may be desirable. In a LLEW device with on-chip heating elements for incubation and for thermocycling (for example, for polymerase chain reaction), the oil may be selected to withstand heating and high temperatures. An oil with sufficiently high dielectric constant may reduce actuation voltage that induces droplet motion.

In LLEW, the solid comprising an oil layer above it may act as an electrical barrier between an electrode array and or underlying electro-mechanics and liquid droplet or bodily fluids. This may also provide the slippery surface for droplet motion and improved interaction with bodily fluids. There are a number of different ways in which the described system may be created. A solid surface may be formed on an electrode array by binding a polymer or other dielectric material as a film. If desired, a non-textured film may be bonded on to the electrode array, and then textured either by laser etching, chemical etching or photolithography techniques, for example. A layer of photosensitive material such as a photoresist (SU-8) may be coated onto the electrode array. The textured solid layer may be covered with lubricating oil by spin-coating, spraying, dip-coating, brushing, drop coating, or by dispensing from a reservoir. The lubricating oil may be kept from flowing out of the surface of the solid (e.g. LLEW) chip by creating physical or chemical barriers at the periphery of the device. The surface of the solid has two unique properties that are desirable for biological sample manipulation or biological implantation. On an electrowetting surface, the actuation voltage may be lowered significantly because the LLEW array has such a smooth surface. Additionally, the LLEW surface architecture may reduce cross-contamination between samples by lowering the trail droplets leave behind as well as improving cleaning mechanism. A nearly molecular level smoothness of oil surface on an LLEW electrode array may reduce or eliminate droplet pinning. A droplet made of an aqueous solution riding on the oil surface may experience little to no drag from the surface and hence have a small difference between the advancing and receding angle—this feature is particularly useful for embodiments wherein the lubricating oil is applied to the surface of a biological implant. The elimination of these two phenomena may result in low actuation voltage. Droplets may be actuated at voltages as low as 1V. In a LLEW device, a droplet riding on a thin layer of oil may never physically come in contact with the solid dielectric substrate below the oil—also particularly useful for embodiments wherein the oil is applied to the surface of a biological implant. This may reduce or eliminate the amount of material left behind and hence cross-contamination between samples that interact with the same spot.

When a LLEW device is contaminated with a solid particle such as dust, a droplet may be maneuvered over the contaminant to remove the contaminant from the liquid film surface as a part of a cleaning routine. This cleaning routine may be further extended to clean the entire surface of electrowetting device. For example, a cleaning routine may be used between two biological experiments on a LLEW microfluidic chip to reduce cross contamination. In some cases, when a droplet stays at a location for a long period of time, a few molecules may diffuse from the droplet into the oil below. Any residue left behind by a droplet through diffusion may also be cleaned with similar washing routines. As droplets are transported on a LLEW device, the droplets may carry and deplete the oil film from the surface. The oil on the surfaces may be replenished by injecting oil from an external reservoir; for example, from an inkjet cartridge, syringe pump or other dispensing mechanisms. The lubricating oil surface may be washed away entirely and replaced with a fresh layer of oil to prevent cross contamination between two consecutive experiments.

As mentioned above, a particular advantage of the instant disclosure is a lowering of the actuation voltage sufficient to perform droplet operations on the surfaces described herein. In some embodiments, the actuation voltage is about 1 V to about 10 V. In some embodiments, the actuation voltage is about 1 V to about 2 V, about 1 V to about 3 V, about 1 V to about 4 V, about 1 V to about 5 V, about 1 V to about 6 V, about 1 V to about 7 V, about 1 V to about 8 V, about 1 V to about 9 V, about 1 V to about 10 V, about 2 V to about 3 V, about 2 V to about 4 V, about 2 V to about 5 V, about 2 V to about 6 V, about 2 V to about 7 V, about 2 V to about 8 V, about 2 V to about 9 V, about 2 V to about 10 V, about 3 V to about 4 V, about 3 V to about 5 V, about 3 V to about 6 V, about 3 V to about 7 V, about 3 V to about 8 V, about 3 V to about 9 V, about 3 V to about 10 V, about 4 V to about 5 V, about 4 V to about 6 V, about 4 V to about 7 V, about 4 V to about 8 V, about 4 V to about 9 V, about 4 V to about 10 V, about 5 V to about 6 V, about 5 V to about 7 V, about 5 V to about 8 V, about 5 V to about 9 V, about 5 V to about 10 V, about 6 V to about 7 V, about 6 V to about 8 V, about 6 V to about 9 V, about 6 V to about 10 V, about 7 V to about 8 V, about 7 V to about 9 V, about 7 V to about 10 V, about 8 V to about 9 V, about 8 V to about 10 V, or about 9 V to about 10 V. In some embodiments, the actuation voltage is about 1 V, about 2 V, about 3 V, about 4 V, about 5 V, about 6 V, about 7 V, about 8 V, about 9 V, or about 10 V. In some embodiments, the actuation voltage is at least about 1 V, about 2 V, about 3 V, about 4 V, about 5 V, about 6 V, about 7 V, about 8 V, or about 9 V. In some embodiments, the actuation voltage is at most about 2 V, about 3 V, about 4 V, about 5 V, about 6 V, about 7 V, about 8 V, about 9 V, or about 10 V. In some embodiments, the actuation voltage is about 10 V to about 100 V. In some embodiments, the actuation voltage is about 10 V to about 20 V, about 10 V to about 30 V, about 10 V to about 40 V, about 10 V to about 50 V, about 10 V to about 60 V, about 10 V to about 70 V, about 10 V to about 80 V, about 10 V to about 90 V, about 10 V to about 100 V, about 20 V to about 30 V, about 20 V to about 40 V, about 20 V to about 50 V, about 20 V to about 60 V, about 20 V to about 70 V, about 20 V to about 80 V, about 20 V to about 90 V, about 20 V to about 100 V, about 30 V to about 40 V, about 30 V to about 50 V, about 30 V to about 60 V, about 30 V to about 70 V, about 30 V to about 80 V, about 30 V to about 90 V, about 30 V to about 100 V, about 40 V to about 50 V, about 40 V to about 60 V, about 40 V to about 70 V, about 40 V to about 80 V, about 40 V to about 90 V, about 40 V to about 100 V, about 50 V to about 60 V, about 50 V to about 70 V, about 50 V to about 80 V, about 50 V to about 90 V, about 50 V to about 100 V, about 60 V to about 70 V, about 60 V to about 80 V, about 60 V to about 90 V, about 60 V to about 100 V, about 70 V to about 80 V, about 70 V to about 90 V, about 70 V to about 100 V, about 80 V to about 90 V, about 80 V to about 100 V, or about 90 V to about 100 V. In some embodiments, the actuation voltage is about 10 V, about 20 V, about 30 V, about 40 V, about 50 V, about 60 V, about 70 V, about 80 V, about 90 V, or about 100 V. In some embodiments, the actuation voltage is at least about 10 V, about 20 V, about 30 V, about 40 V, about 50 V, about 60 V, about 70 V, about 80 V, or about 90 V. In some embodiments, the actuation voltage is at most about 20 V, about 30 V, about 40 V, about 50 V, about 60 V, about 70 V, about 80 V, about 90 V, or about 100 V. In some embodiments, the actuation voltage is about 125 V to about 400 V. In some embodiments, the actuation voltage is about 125 V to about 150 V, about 125 V to about 175 V, about 125 V to about 200 V, about 125 V to about 225 V, about 125 V to about 250 V, about 125 V to about 275 V, about 125 V to about 300 V, about 125 V to about 325 V, about 125 V to about 350 V, about 125 V to about 375 V, about 125 V to about 400 V, about 150 V to about 175 V, about 150 V to about 200 V, about 150 V to about 225 V, about 150 V to about 250 V, about 150 V to about 275 V, about 150 V to about 300 V, about 150 V to about 325 V, about 150 V to about 350 V, about 150 V to about 375 V, about 150 V to about 400 V, about 175 V to about 200 V, about 175 V to about 225 V, about 175 V to about 250 V, about 175 V to about 275 V, about 175 V to about 300 V, about 175 V to about 325 V, about 175 V to about 350 V, about 175 V to about 375 V, about 175 V to about 400 V, about 200 V to about 225 V, about 200 V to about 250 V, about 200 V to about 275 V, about 200 V to about 300 V, about 200 V to about 325 V, about 200 V to about 350 V, about 200 V to about 375 V, about 200 V to about 400 V, about 225 V to about 250 V, about 225 V to about 275 V, about 225 V to about 300 V, about 225 V to about 325 V, about 225 V to about 350 V, about 225 V to about 375 V, about 225 V to about 400 V, about 250 V to about 275 V, about 250 V to about 300 V, about 250 V to about 325 V, about 250 V to about 350 V, about 250 V to about 375 V, about 250 V to about 400 V, about 275 V to about 300 V, about 275 V to about 325 V, about 275 V to about 350 V, about 275 V to about 375 V, about 275 V to about 400 V, about 300 V to about 325 V, about 300 V to about 350 V, about 300 V to about 375 V, about 300 V to about 400 V, about 325 V to about 350 V, about 325 V to about 375 V, about 325 V to about 400 V, about 350 V to about 375 V, about 350 V to about 400 V, or about 375 V to about 400 V. In some embodiments, the actuation voltage is about 125 V, about 150 V, about 175 V, about 200 V, about 225 V, about 250 V, about 275 V, about 300 V, about 325 V, about 350 V, about 375 V, or about 400 V. In some embodiments, the actuation voltage is at least about 125 V, about 150 V, about 175 V, about 200 V, about 225 V, about 250 V, about 275 V, about 300 V, about 325 V, about 350 V, or about 375 V. In some embodiments, the actuation voltage is at most about 150 V, about 175 V, about 200 V, about 225 V, about 250 V, about 275 V, about 300 V, about 325 V, about 350 V, about 375 V, or about 400 V.

In some embodiments, the film layer comprises one or more polymeric films inorganic films, composite films, or combinations thereof. In some embodiments, the film layer is a composite film layer comprising two of more films laminated together. In some embodiments, the film layer is a composite film layer comprising three of more films laminated together. In some embodiments, the film layer is a composite film layer comprising four or more films laminated together. In some embodiments, the film layer is a composite film layer comprising five of more films laminated together. The composite films take advantage of the various properties of each material used.

In some embodiments, the film layer may comprise insulating dielectric materials. In some embodiments, the film layer comprises polyethylene, polypropylene, polystyrene, polyetheretherketone (PEEK), polyimide, polyacetal, polysulfone, polyphenylene ether, polyphenylene Sulfide (PPS), polyvinyl chloride, synthetic rubber, natural rubber, neoprene, nylon, polyacrylonitrile, polyvinyl butyral, silicone, parafilm, polyethylene terephthalate, polybutylene terephthalate, polyamides, polyoxymethlyene, polycarbonate, polymethylpentene, polyphenylene oxide (Polyphenyl ether), polyphthalamide (PPA), polylactic acid, synthetic cellulose ethers (e.g., methyl cellulose, ethyl cellulose, propyl cellulose, hydroxyethyl cellulose, hydroxyethyl cellulose, hydroxypropyl cellulose (HPC), hydroxyethyl methyl cellulose, hydroxypropyl methyl cellulose (HPMC), ethyl hydroxyethyl cellulose), paraffins, microcrystalline wax, epoxy, polytetrafluoroethylene (PTFE), tetrafluoroethylene (TFE), fluorinated ethylenepropylene copolymer (FEP), polyvinylidene fluoride (PVDF), perfluoroalkoxytetrafluoroethylene copolymer (PFA), perfluoromethyl vinylether copolymer (MFA), ethylenechlorotrifluoroethylene copolymer (ECTFE), ethylene-tetrafluoroethylene copolymer (ETFE), perfluoropolyether (PFPE), polychlorotetrafluoroethylene (PCTFE), ceramic, borosilicate glass, quartz, alumina, silica, clay minerals, bentonite, kaolinite, vermiculite, graphite, molybdenum disulfide, mica, boron nitride, sodium formate, sodium oleate, sodium palmitate, sodium sulfate, sodium alginate, other polymer materials, other ceramic materials, or combinations thereof.

In some embodiments, the film layer may be modified. In some embodiments, the film layer may be modified by either applying a secondary coating to the film layer or by functionalizing the surface of the film layer. Either the secondary coating or surface functionalization may be selected to improve the affinity of the film layer for the liquid layer. Modification of the film layer may be accomplished in either the liquid phase or gas phase. The film layer may be modified on either side, and both sides of the film layer may comprise the same or different modifications. The film layer may be modified to improve hydrophobicity and fluid manipulation.

In some embodiments, the modifications may be selected to improve other properties such as durability, dielectric breakdown, electric resistivity, dielectric constant, environmental impact, elasticity, coefficient of thermal expansion, thermal conductivity, or combinations thereof.

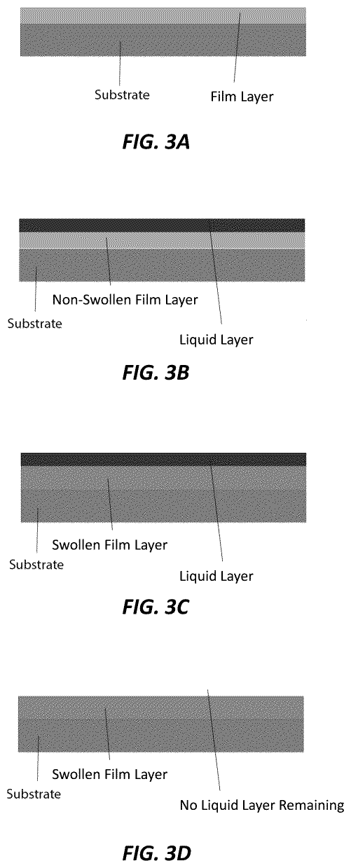

In some embodiments, the liquid layer may diffuse into the film layer causing the film layer to swell. FIGS. 3A-3D are illustrations depicting swelling of the film layer by the liquid layer. Because the film layer is smooth, the liquid layer does not fill the any surface

In some embodiments, the liquid layer is non-uniform. In some embodiments, the liquid layer has an average initial thickness from about 0.1 μm to about 500 μm. In some embodiments, the liquid layer has a thickness of at least about 0.1 μm, 0.2 μm, 0.3 μm, 0.4 μm, 0.5 μm, 0.6 μm, 0.7 μm, 0.8 μm, 0.9 μm, 1 μm, 2 μm, 3 μm, 4 μm, 5 μm, 6 μm, 7 μm, 8 μm, 9 μm, 10 μm, 20 μm, 30 μm, 40 μm, 50μ, 60 μm, 70μ, 80 μm, 90μ, 100μ, 200μ, 300μ, 400μ, 500 μm, or any values therebetween. In some embodiments, the liquid layer has a thickness of at most about 500 μm, 400 μm, 300 μm, 200 μm, 100 μm, 90 μm, 80 μm, 70 μm, 60 μm, 50 μm, 40 μm, 30 μm, 20 μm, 10 μm, 9 μm, 8 μm, 7 μm, 6 μm, 5 μm, 4 μm, 3 μm, 2 μm, 1 μm, 0.9 μm, 0.8 μm, 0.7 μm, 0.6 μm, 0.5 μm, 0.4 μm, 0.3 μm, 0.2 μm, 0.1 μm, or any values therebetween. In some embodiments, the liquid layer has a thickness of at most about 0.1 centimeters (cm), 0.2 cm, 0.3 cm, 0.4 cm, 0.5 cm, 0.6 cm, 0.7 cm, 0.8 cm, 0.9 cm, 1.0 cm, 1.1 cm, 1.2 cm, 1.3 cm, 1.4 cm, 1.5 cm, 1.6 cm, 1.7 cm, 1.8 cm, 1.9 cm, or 2.0 cm. In embodiments when the liquid layer is non-uniform, the liquid layer may have a minimum thickness (e.g., initially, after some passage of time, under a droplet, etc.) of about 1 nanometer (nm), 2 nm, 3 nm, 4 nm, 5 nm, 6 nm, 7 nm, 8 nm, 9 nm, 10 nm, 20 nm, 30 nm, 40 nm 50 nm, 60 nm, 70 nm, 80 nm, 90 nm, 100 nm, 200 nm, 300 nm, 400 nm, 500 nm, 600 nm, 700 nm, 800 nm, 900 nm, 1000 nm, 2000 nm, 3000 nm, 4000 nm, 5000 nm, 6000 nm, 7000 nm, 8000 nm, 9000 nm, 10000 nm, etc. In some embodiments, the thickness of the liquid layer may decrease over time after application of the liquid layer. In some embodiments, the thickness of the liquid layer may decrease (e.g., locally) under weight of a droplet atop the liquid layer.

The viscosity of the liquid layer may be selected to optimize fluid mobility, reduce drag, and increase durability of the liquid layer. In some embodiments, the liquid layer has a viscosity of about 0.5 centistokes (cSt) to about 100 cSt. In some embodiments, the liquid layer has a viscosity of about 0 cSt to about 20 cSt. In some embodiments, the liquid layer has a viscosity of about 0 cSt to about 30 cSt. In some embodiments, the liquid layer has a viscosity of about 5 cSt to about 20 cSt. In some embodiments, the liquid layer has a viscosity of at least about 0 cSt, 0.1 cSt, 0.2 cSt, 0.3 cSt, 0.4 cSt, 0.5 cSt, 0.6 cSt, 0.7 cSt, 0.8 cSt, 0.9 cSt, 1 cSt, 2 cSt, 3 cSt, 4 cSt, 5 cSt, 6 cSt, 7 cSt, 8 cSt, 9 cSt, 10 cSt, 20 cSt, 30 cSt, 40 cSt, 50 cSt, 60 cSt, 70 cSt, 80 cSt, 90 cSt, 100 cSt, or any values therebetween. In some embodiments, the liquid layer has a viscosity of at most about 100 cSt, 90 cSt, 80 cSt, 70 cSt, 60 cSt, 50 cSt, 40 cSt, 30 cSt, 20 cSt, 10 cSt, 9 cSt, 8 cSt, 7 cSt, 6 cSt, 5 cSt, 4 cSt, 3 cSt, 2 cSt, 1 cSt, 0.9 cSt, 0.8 cSt, 0.7 cSt, 0.6 cSt, 0.5 sCt, or any values therebetween. Values provided herein for viscosity of the liquid layer may be measured when the liquid layer is at room temperature.

In some embodiments, the liquid layer has a viscosity of about 0 cST to about 5 cST. In some embodiments, the liquid layer has a viscosity of about 0 cST to about 0.5 cST, about 0 cST to about 1 cST, about 0 cST to about 1.5 cST, about 0 cST to about 2 cST, about 0 cST to about 2.5 cST, about 0 cST to about 3 cST, about 0 cST to about 3.5 cST, about 0 cST to about 4 cST, about 0 cST to about 4.5 cST, about 0 cST to about 5 cST, about 0.5 cST to about 1 cST, about 0.5 cST to about 1.5 cST, about 0.5 cST to about 2 cST, about 0.5 cST to about 2.5 cST, about 0.5 cST to about 3 cST, about 0.5 cST to about 3.5 cST, about 0.5 cST to about 4 cST, about 0.5 cST to about 4.5 cST, about 0.5 cST to about 5 cST, about 1 cST to about 1.5 cST, about 1 cST to about 2 cST, about 1 cST to about 2.5 cST, about 1 cST to about 3 cST, about 1 cST to about 3.5 cST, about 1 cST to about 4 cST, about 1 cST to about 4.5 cST, about 1 cST to about 5 cST, about 1.5 cST to about 2 cST, about 1.5 cST to about 2.5 cST, about 1.5 cST to about 3 cST, about 1.5 cST to about 3.5 cST, about 1.5 cST to about 4 cST, about 1.5 cST to about 4.5 cST, about 1.5 cST to about 5 cST, about 2 cST to about 2.5 cST, about 2 cST to about 3 cST, about 2 cST to about 3.5 cST, about 2 cST to about 4 cST, about 2 cST to about 4.5 cST, about 2 cST to about 5 cST, about 2.5 cST to about 3 cST, about 2.5 cST to about 3.5 cST, about 2.5 cST to about 4 cST, about 2.5 cST to about 4.5 cST, about 2.5 cST to about 5 cST, about 3 cST to about 3.5 cST, about 3 cST to about 4 cST, about 3 cST to about 4.5 cST, about 3 cST to about 5 cST, about 3.5 cST to about 4 cST, about 3.5 cST to about 4.5 cST, about 3.5 cST to about 5 cST, about 4 cST to about 4.5 cST, about 4 cST to about 5 cST, or about 4.5 cST to about 5 cST. In some embodiments, the liquid layer has a viscosity of about 0 cST, about 0.5 cST, about 1 cST, about 1.5 cST, about 2 cST, about 2.5 cST, about 3 cST, about 3.5 cST, about 4 cST, about 4.5 cST, or about 5 cST. In some embodiments, the liquid layer has a viscosity of about at least about 0 cST, about 0.5 cST, about 1 cST, about 1.5 cST, about 2 cST, about 2.5 cST, about 3 cST, about 3.5 cST, about 4 cST, or about 4.5 cST. In some embodiments, the liquid layer has a viscosity of about at most about 0.5 cST, about 1 cST, about 1.5 cST, about 2 cST, about 2.5 cST, about 3 cST, about 3.5 cST, about 4 cST, about 4.5 cST, or about 5 cST. In some embodiments, the liquid layer has a viscosity of about 5 cST to about 20 cST. In some embodiments, the liquid layer has a viscosity of about 5 cST to about 7.5 cST, about 5 cST to about 10 cST, about 5 cST to about 12.5 cST, about 5 cST to about 15 cST, about 5 cST to about 17.5 cST, about 5 cST to about 20 cST, about 7.5 cST to about 10 cST, about 7.5 cST to about 12.5 cST, about 7.5 cST to about 15 cST, about 7.5 cST to about 17.5 cST, about 7.5 cST to about 20 cST, about 10 cST to about 12.5 cST, about 10 cST to about 15 cST, about 10 cST to about 17.5 cST, about 10 cST to about 20 cST, about 12.5 cST to about 15 cST, about 12.5 cST to about 17.5 cST, about 12.5 cST to about 20 cST, about 15 cST to about 17.5 cST, about 15 cST to about 20 cST, or about 17.5 cST to about 20 cST. In some embodiments, the liquid layer has a viscosity of about 5 cST, about 7.5 cST, about 10 cST, about 12.5 cST, about 15 cST, about 17.5 cST, or about 20 cST. In some embodiments, the liquid layer has a viscosity of about at least about 5 cST, about 7.5 cST, about 10 cST, about 12.5 cST, about 15 cST, or about 17.5 cST. In some embodiments, the liquid layer has a viscosity of about at most about 7.5 cST, about 10 cST, about 12.5 cST, about 15 cST, about 17.5 cST, or about 20 cST. In some embodiments, the liquid layer has a viscosity of about 20 cST to about 100 cST. In some embodiments, the liquid layer has a viscosity of about 20 cST to about 30 cST, about 20 cST to about 40 cST, about 20 cST to about 50 cST, about 20 cST to about 60 cST, about 20 cST to about 70 cST, about 20 cST to about 80 cST, about 20 cST to about 90 cST, about 20 cST to about 100 cST, about 30 cST to about 40 cST, about 30 cST to about 50 cST, about 30 cST to about 60 cST, about 30 cST to about 70 cST, about 30 cST to about 80 cST, about 30 cST to about 90 cST, about 30 cST to about 100 cST, about 40 cST to about 50 cST, about 40 cST to about 60 cST, about 40 cST to about 70 cST, about 40 cST to about 80 cST, about 40 cST to about 90 cST, about 40 cST to about 100 cST, about 50 cST to about 60 cST, about 50 cST to about 70 cST, about 50 cST to about 80 cST, about 50 cST to about 90 cST, about 50 cST to about 100 cST, about 60 cST to about 70 cST, about 60 cST to about 80 cST, about 60 cST to about 90 cST, about 60 cST to about 100 cST, about 70 cST to about 80 cST, about 70 cST to about 90 cST, about 70 cST to about 100 cST, about 80 cST to about 90 cST, about 80 cST to about 100 cST, or about 90 cST to about 100 cST. In some embodiments, the liquid layer has a viscosity of about 20 cST, about 30 cST, about 40 cST, about 50 cST, about 60 cST, about 70 cST, about 80 cST, about 90 cST, or about 100 cST. In some embodiments, the liquid layer has a viscosity of about at least about 20 cST, about 30 cST, about 40 cST, about 50 cST, about 60 cST, about 70 cST, about 80 cST, or about 90 cST. In some embodiments, the liquid layer has a viscosity of about at most about 30 cST, about 40 cST, about 50 cST, about 60 cST, about 70 cST, about 80 cST, about 90 cST, or about 100 cST. Values provided herein for viscosity of the liquid layer may be measured when the liquid layer is at room temperature.

In some embodiments, the liquid layer has a static contact angle with the film layer of about 10 degrees or less. The small static contact angle helps to improve lubricity and reduces fouling and pinning during fluid manipulation. In some embodiments, the liquid layer has a static contact angle with the film layer of at least about 0.1 degrees, 0.2 degrees, 0.3 degrees, 0.4 degrees, 0.5 degrees, 0.6 degrees, 0.7 degrees, 0.8 degrees, 0.9 degrees, 1 degree, 2 degrees, 3 degrees, 4 degrees, 5 degrees, 6 degrees, 7 degrees, 8 degrees, 9 degrees, 10 degrees, or any values therebetween. In some embodiments, the liquid layer has a static contact angle with the film layer of at most about 10 degrees, 9 degrees, 8 degrees, 7 degrees, 6 degrees, 5 degrees, 4 degrees, 3 degrees, 2 degrees, 1 degree, 0.9 degrees, 0.8 degrees, 0.7 degrees, 0.6 degrees, 0.5 degrees, 0.4 degrees, 0.3 degrees, 0.2 degrees, 0.1 degrees, or any values therebetween.

In some embodiments the liquid layer is a lubricating layer. The lubricating layer improves overall fluid mobility by reducing friction between the film layer and the droplet, preventing droplet pinning, reducing fouling, and reducing contact angle hysteresis. Good fluid mobility may be defined by the ability to move a fluid accurately, reliably, at a high speed, for extended periods of time, or without pinning and fouling on the surface.

In some embodiments, the lubricating layer is selected for its affinity for the film layer and its immiscibility with the manipulated fluid. The affinity of the liquid layer for the film layer prevents the liquid layer from being displaced by the manipulated fluid. Because the lubricating layer is selected for its affinity to the film layer, the surface coating is easier to manufacture. For example, hydrocarbon fluids may be used with polyolefin films, silicone fluids may be used with silicone films, and fluorinated fluids may be used with fluorinated polymer films.

In some embodiments, the lubricating layer is a hydrocarbon layer, a silicone layer, a fluorinated layer, or combinations thereof.

In some embodiments, the lubricating layer comprises polydimethylsiloxanes, polymethyl hydrogen siloxane/hydrogen silicone oil, amino silicone oil, phenyl methyl silicone oil, diphenyl silicone oil, vinyl silicone oil, hydroxy silicone oil, cyclosiloxanes, polyalkylene oxide silicones, silicone resins, perfluoropolyether (PFPE), perfluoroalkanes, fluorinated ionic fluid, fluorinated silicone oils, perfluoroalkylether, perfluoro tri-n-butylamine (FC-40), hydrofluoroether (HFE) liquids, ionic liquids, mineral oils, ferrofluids, polyphenyl ether, vegetable oil, esters of saturated fatty and dibasic acids, grease, fatty acids, triglycerides, polyalphaolefin, polyglycol hydrocarbons, other alkanes, other non-hydrocarbon synthetic oils, or combinations thereof.

In some embodiments, the lubricating layer may comprise an additive. In some embodiments, the additive is a rheology modifier, filler, solvent, surfactant, dye, or combinations thereof. Rheology modifiers, fillers, and solvents, Rheology modifiers, fillers and solvents may help tune the viscosity of the liquid and can give non-Newtonian flow properties to the liquid. Fillers may help improve material properties such as thermal conductivity of dielectric constant and may also change rheological properties. Surfactants and solvents can help tune the surface energy of the lubricating layer with the film layer and the manipulated fluid.

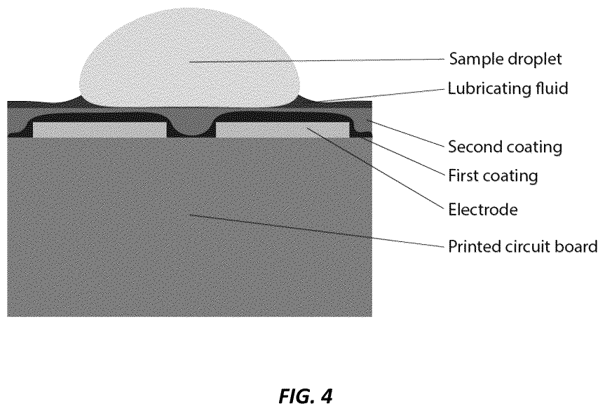

In some embodiments, a part of, or all of the surface coating may be removed and replaced. In some embodiments, the surface coating may be used once. In some embodiments, the surface coating may be used multiple times. In some embodiments, the coating may be permanent. FIG. 4 is an illustration depicting a permanent surface coating for fluid manipulation, according to some embodiments.

In some embodiments, the coatings described herein may be applied to a surface intended to contact a fluid. In some embodiments, non-limiting examples of a surface intended to contact a fluid may include a cannula, connector, catheter (e.g., central line, peripherally inserted central catheter (PICC) line, urinary, vascular, peritoneal dialysis, and central venous catheters), catheter connector (e.g., Leur-Lok and needleless connectors), clamp, skin hook, cuff, retractor, shunt, needle, capillary tube, endotracheal tube, ventilator, associated ventilator tubing, drug delivery vehicle, syringe, microscope slide, plate, film, laboratory work surface, well, well plate, Petri dish, tile, jar, flask, beaker, vial, test tube, tubing connector, column, container, cuvette, bottle, drum, vat, tank, organ, organ implant, or organ component (e.g., intrauterine device, defibrillator, corneal, breast, knee replacement, and hip replacement implants), artificial organ or a component thereof (e.g., heart valve, ventricular assist devices, total artificial hearts, cochlear implant, visual prosthetic, and components thereof), dental tool, dental implant (e.g., root form, plate form, and subperiosteal implants), biosensor (e.g., glucose and insulin monitor, blood oxygen sensor, hemoglobin sensor, biological microelectromechanical devices (bioMEMs), sepsis diagnostic sensor, and other protein and enzyme sensors), bioelectrode, endoscope (hysteroscope, cystoscope, amnioscope, laparoscope, gastroscope, mediaslinoscope, bronchoscope, esophagoscope, rhinoscope, arthroscope, proctoscope, colonoscope, nephroscope, angioscope, thoracoscope, esophagoscope, laryngoscope, and encepbaloscope) wound dressing (e.g., bandages, sutures, staples), flow cell, microfluidic device, and combinations thereof.

In some embodiments, the coatings described herein may be applied to a device, such as research and diagnostic arrays. Examples of research and diagnostic arrays may include sample preparation, amplification, rolling circle amplification, bridge amplification, sequencing, circular consensus sequencing, next generation sequencing, polymerase chain reaction, enzymatic polymer synthesis, and sample detection arrays.

### Fluid Manipulation Apparatus

In another aspect, the present disclosure provides an apparatus for fluid manipulation comprising the surface coatings described herein. In some embodiments, the apparatus may be an electrowetting microfluidic device.

In some embodiments, the apparatus includes a substrate having one or more electrodes and a first surface comprising the coatings described herein. In some embodiments, the first surface comprises a film layer and a liquid layer.

In some embodiments, the electrodes consist of conductive plates that charge electrically to actuate the fluid. In some embodiments, the electrodes may be arranged in an arbitrary layout, for example as a rectangular grid, or a collection of discrete paths. In some embodiments, the electrodes may have an arbitrary shape.

In some embodiments, the electrodes comprise a conductive metal, conductive oxide, semiconductors, conductive polymers, or combinations thereof. In some embodiments, the electrodes are made of a conductive metal selected from: gold, silver, copper, nickel, aluminum, platinum, titanium, or combinations thereof. In some embodiments, the electrodes comprise a conductive oxide selected from: indium tin oxide, aluminum doped zinc oxide, and combinations thereof. In some embodiments, the electrodes comprise a semiconductor, such as silicon dioxide. Because the electrodes are conductive, a first surface comprising an insulator or dielectric is used to separate the electrodes and the manipulated fluid to prevent oxidation and reduction reactions on the electrodes and the manipulated fluid. In some embodiments, the dielectric may be up to about 1 millimeters (mm). For example, the dielectric may be up to about 0.001 mm, 0.002 mm, 0.003 mm, 0.004 mm, 0.005 mm, 0.006 mm, 0.007 mm, 0.008 mm, 0.009 mm, 0.01 mm, 0.02 mm, 0.03 mm, 0.04 mm, 0.05 mm, 0.06 mm, 0.07 mm, 0.08 mm, 0.09 mm, 0.1 mm 0.2 mm, 0.3 mm, 0.4 mm, 0.5 mm, 0.6 mm, 0.7 mm, 0.8 mm, 0.9 mm, or 1 mm. In some embodiments, the dielectric may be up to about 2 mm, 3 mm, 4 mm, 5 mm, 6 mm, 7 mm, 8 mm, 9 mm, or 10 mm.

In some embodiments, the substrate supporting the electrodes may comprise any insulating materials of any thickness and rigidity.

In some embodiments, the electrodes are fabricated on standard rigid and flexible printed circuit board (“PCB”) substrates. In some embodiments, the substrate for the PCB is FR4 (glass-epoxy), FR2 (glass-epoxy) or insulated metal substrate (IMS), polyimide film (example commercial brands include Kapton, Pyralux), polyethylene terapthalate (PET), ceramic, or other commercially available substrates.

In some embodiments, the substrate further comprises a sealant layer. In some embodiments, the sealant layer may act as an insulator, moisture barrier, corrosion barrier, or reaction barrier. In some embodiments, sealant layer comprises fluoropolymers, polyurethanes, acrylics, silicones, polyolefins, parylenes, or combinations thereof.

In some embodiments, the electrodes are fabricated using thin film transistor (TFT) technology. A TFT may be a type of field-effect transistor (FET) where the transistor of the TFT is made via thin film deposition. TFTs may be grown on a supporting (but non-conducting) substrate (e.g., glass), which differs from bulk metal oxide field effect transistor (MOSFET), where the semiconductor material typically is the substrate (e.g., a silicon wafer).

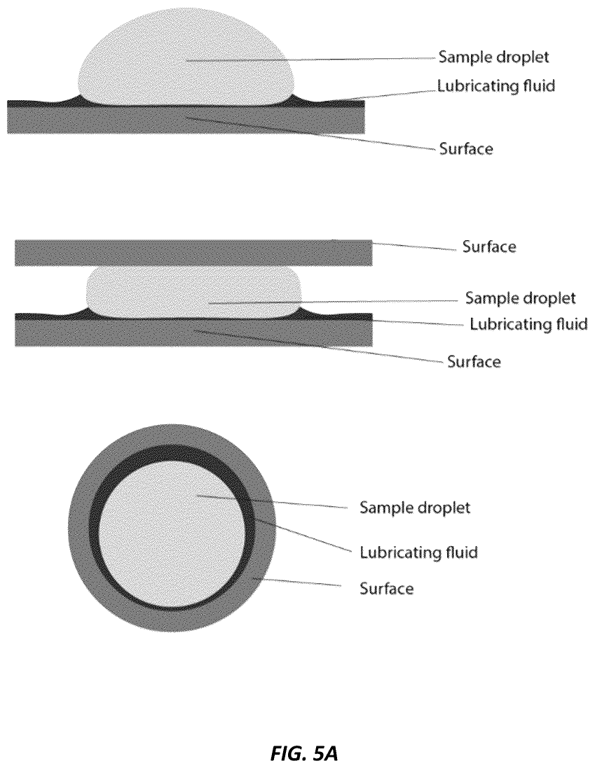

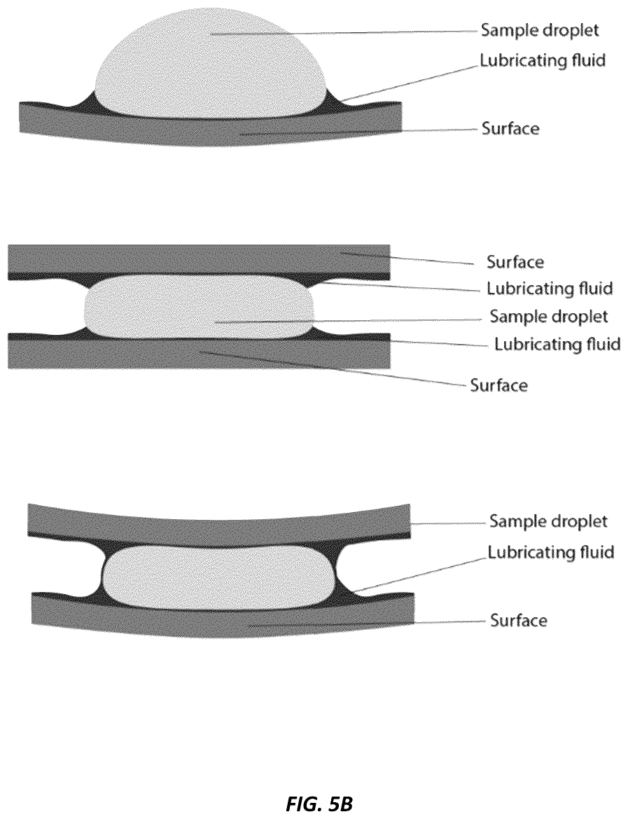

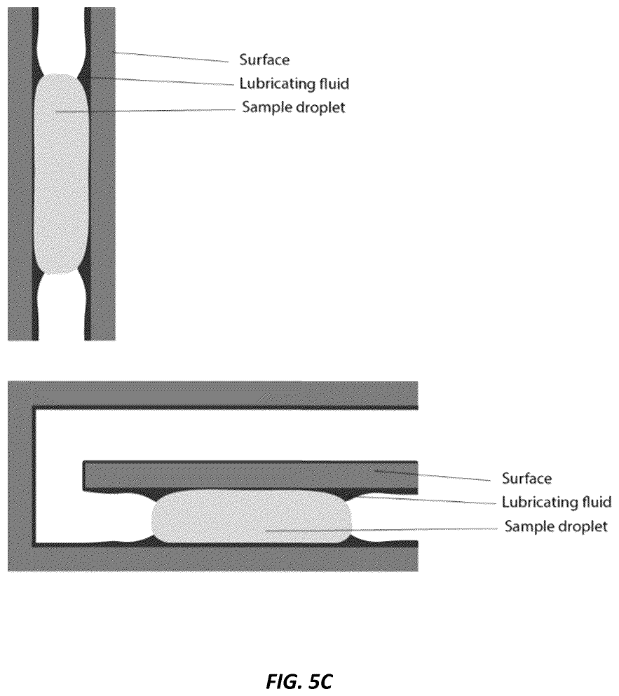

As illustrated in FIGS. 5A-5C, the first surface of the apparatus may be flat, curved, tubular, horizontal, vertical, or any combinations thereof. As illustrated in FIGS. 5A-5C, the apparatus may further comprise a second surface parallel to the first surface. In some embodiments, the second surface comprises the surface coatings described herein. As illustrated in FIGS. 5A-5C, the manipulated fluid may be positioned between the first surface and the second surface. In some embodiments, the surface coating may be used on the surface of an opto-electrowetting device or other optical manipulation technology, such as laser tweezers.

Printed circuit boards (PCBs) manufactured by typical processes have surface roughness in the form of: canyons (gaps) between electrodes, holes for establishing connection between multiple layers (also known as vias), holes to solder through-hole components and any other imperfections from manufacturing errors, and the like. Accordingly, a gap-filling liquid may be used to smooth the surface of the PCB and promote adhesion between the substrate and the first surface.

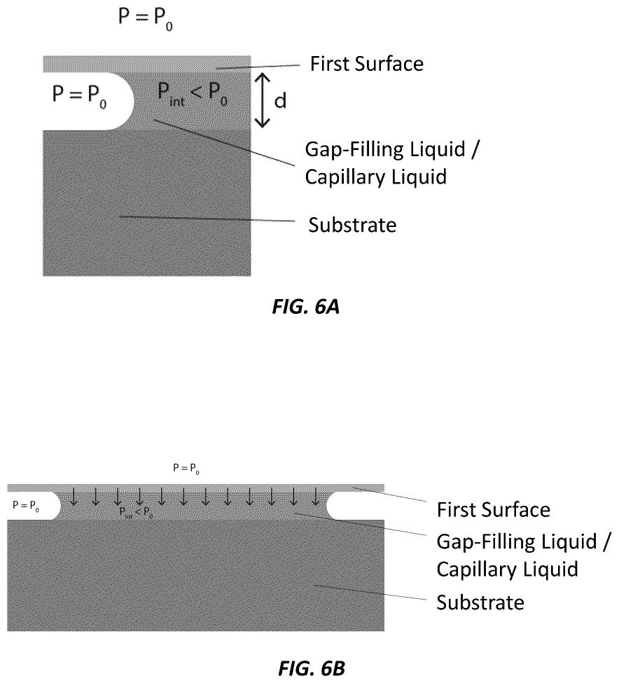

In some embodiments, the apparatus further comprises a gap-filling liquid between the substrate and the first surface. The gap-filling liquid helps to fill air gaps between the substrate and the first surface. FIGS. 6A and 6B are illustrations depicting the relationship between the substrate, the capillary fluid, and the first surface.

In some embodiments, the gap-filling liquid forms a displaced volume between the substrate and the first surface. In some embodiments, the displaced volume has an average height of least about 0.01 μm, 0.02 μm, 0.03 μm, 0.04 μm, 0.05 μm, 0.06 μm, 0.07, 0.08 μm, 0.09 μm, 0.1 μm, 0.2 μm, 0.3 μm, 0.4 μm, 0.5 μm, 0.6 μm, 0.7 μm, 0.8 μm, 0.9 μm, 1 μm, 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 200 μm, 300 μm, 400 μm, 500 μm, or any values therebetween. In some embodiments, the displaced volume has an average height of at most about 500 μm, 400 μm, 300 μm, 200 μm, 100 μm, 90 μm, 80 μm, 70 μm, 60 μm, 50 μm, 40 μm, 30 μm, 20 μm, 10 μm, 9 μm, 8 μm, 7 μm, 6 μm, 5 μm, 4 μm, 3 μm, 2 μm, 1 μm, 0.9 μm, 0.8 μm, 0.7 μm, 0.6 μm, 0.5 μm, 0.4 μm, 0.3 μm, 0.2 μm, 0.1 μm, 0.09 μm, 0.08 μm, 0.07 μm, 0.06 μm, 0.05 μm, 0.04 μm, 0.03 μm, 0.02 μm, 0.01 μm, or any values therebetween.

The gap-filling liquid may serve multiple purposes. First, in an electrowetting device, the gap-filling liquid may increase electrowetting force by replacing a low dielectric constant medium, such as air, with a higher dielectric constant liquid. Switching from a low dielectric constant medium to a higher dielectric constant medium increases the capacitance between the electrodes and the manipulated fluid, therefore increasing the charges in both the electrodes and the manipulated fluid and increasing the applied force. Second, by replacing air, the gap-filling fluid reduces the thermal expansion and improves heat transfer between the substrate and the first surface thereby allowing faster heating and cooling of the manipulated fluid surface and reduced expansion and contraction of the of the area under the film layer. Thirdly, the gap-filling fluid provides adhesive properties.

In some embodiments, the gap-filling liquid is a gel, paste, grease, high viscosity oil, a low viscosity oil, or combinations thereof. In some embodiments, the gap-filling liquid is a silicone paste, lithium grease, silicone grease, thermal paste, dyed grease, or combinations thereof.

In some embodiments, the gap-filling liquid is a capillary liquid, wherein a capillary liquid is a liquid capable of wetting both the first surface and the substrate creating a capillary pressure that decreases the distance between the substrate and the first surface. The decreased distance between the substrate and the first surface further increases the capillary pressure, therefore further decreasing the distance between the substrate and the first surface. This wetting behavior causes the first surface to lie flat and uniformly over the substrate and improves various properties of the apparatus as described above. The capillary pressure may be defined as

$${P}_{int} = {P}_{0} - 2 * γ * \frac{cos θ}{d}$$

where P₀ is atmospheric pressure, gamma y is the surface energy of the capillary liquid, θ is the contact angle of the capillary liquid with the substrate and first surface, and d is the thickness of the capillary liquid at the edge of the liquid line as shown in FIGS. 6A and 6B. In practice, θ is close to zero and the net pressure applied on the first surface is approximately

$$2 * γ * \frac{1}{d} .$$

To provide good adhesion, the capillary liquid should have a contact angle less than 90 degrees with both the substrate and the first surface.

In some embodiments, the capillary liquid has a contact angle with the substrate of less than about 90 degrees. In some embodiments, the capillary liquid has a contact angle with the substrate or sealant layer of at least about 1 degree, 2 degrees, 3 degrees, 4 degrees, 5 degrees, 6 degrees, 7 degrees, 8 degrees, 9 degrees, 10 degrees, 20 degrees, 30 degrees, 40 degrees, 50 degrees, 60 degrees, 70 degrees, 80 degrees, 90 degrees, or any values therebetween. In some embodiments, the capillary liquid has a contact angle with the substrate of at most about 90 degrees, 80 degrees, 70 degrees, 60 degrees, 50 degrees, 40 degrees, 30 degrees, 20 degrees, 10 degrees, 9 degrees, 8 degrees, 7 degrees, 6 degrees, 5 degrees, 4 degrees, 3 degrees, 2 degrees, 1 degree, or any values therebetween.

In some embodiments, the capillary liquid has a contact angle with the first surface of less than about 90 degrees. In some embodiments, the capillary liquid has a contact angle with the first surface of at least about 1 degree, 2 degrees, 3 degrees, 4 degrees, 5 degrees, 6 degrees, 7 degrees, 8 degrees, 9 degrees, 10 degrees, 20 degrees, 30 degrees, 40 degrees, 50 degrees, 60 degrees, 70 degrees, 80 degrees, 90 degrees, or any values therebetween. In some embodiments, the capillary liquid has a contact angle with the first surface of at most about 90 degrees, 80 degrees, 70 degrees, 60 degrees, 50 degrees, 40 degrees, 30 degrees, 20 degrees, 10 degrees, 9 degrees, 8 degrees, 7 degrees, 6 degrees, 5 degrees, 4 degrees, 3 degrees, 2 degrees, 1 degree, or any values therebetween. In some embodiments, a secondary coating may be applied to the first surface to lower the contact angle between the capillary fluid and the first surface.

In some embodiments, the capillary liquid is a hydrocarbon oil, silicone oil, fluorinated oil, liquid acrylate, or combinations thereof. In some embodiments, the capillary liquid is a hydrocarbon oil selected from: mineral oils, medium chain alkanes, polyalphaolefins, and combinations thereof. In some embodiments, the capillary liquid is a silicone oil selected from: methylsilicone oils, methylphenyl silicone oils, fluorosilicone oils, other silicone oils, or combinations thereof. In some embodiments, the capillary liquid is a fluorinated oil selected from: perfluoropolyether, fluoroacrylate, or combinations thereof.

In some embodiments, the gap-filling liquid further comprises at least one additive. In some embodiments, the at least one additive is a rheology modifier, filler, solvent, surfactant, dye, or combinations thereof. In some embodiments, the additive enhances the performance of the gap-filling liquid. Rheology modifiers, fillers and solvents may help tune the viscosity of the liquid and can give non-Newtonian flow properties to the liquid. Fillers may help improve material properties such as thermal conductivity or dielectric constant. Dyes may help with bubble detection after application of the gap-filling liquid.

In some embodiments, the substrate may be modified. In some embodiments, the substrate may be modified by either applying a secondary coating to the substrate or by functionalizing the surface of the substrate. Either the secondary coating or surface functionalization may be selected to improve the affinity of the substrate for the gap-filling liquid.

In some embodiments, the modifications may be selected to provide other properties such as durability, dielectric breakdown, electric resistivity, dielectric constant, environmental impact, elasticity, coefficient of thermal expansion, thermal conductivity, or combinations thereof.

In some embodiments, the substrate comprises a secondary coating selected from: parylene, other vapor deposition coatings, fluoropolymers, polyurethanes, acrylics, silicones, polyolefins, or combinations thereof. In some embodiments, the substrate comprises a functionalized surface selected from: silanes, other chemical vapor deposition precursors, other physical vapor deposition precursors, or combinations thereof.

In some embodiments, the first surface may be modified. In some embodiments, the first surface may be modified by either applying a secondary coating to the film layer or by functionalizing the surface of the film layer. Either the secondary coating or surface functionalization may be selected to improve the affinity of the first surface for the gap-filling liquid.

In some embodiments, the gap-filling liquid is either an insulating or conductive liquid.

In some embodiments, the apparatus further comprises a vacuum between said substrate and said first surface. In some embodiments, the vacuum may achieve the same properties as the gap-filling liquid.

In some embodiments, the apparatus may further comprise a film-frame configured to support the first surface. In some embodiments, the film-frame is configured to maintain or generate tension on the film layer of the first surface. In some embodiments, the film-frame is configured to generate a vacuum pressure between the between the substrate and the first surface.

In some embodiments, the film-frame includes a fluid dispensing unit. In some embodiments, the frame if configured to dispense the liquid layer.

In some embodiments, the film-frame is attached to the first surface at the periphery of the first surface. In some embodiments, the first surface is attached to the film-frame using an adhesive. In some embodiments, the adhesive is a wet adhesive or a dry adhesive. In some embodiments, the adhesive is a thermal adhesive. These adhesive strategies can be selectively implemented in regions (e.g., along the periphery of the frame) or across the entire surface of the films.

In some embodiments, the first surface comprises a working area of about 0.0001 cm² to about 10000 cm². The working area is the area of the first surface configured to contact the fluid to be manipulated. In some embodiments, the first surface comprises a working area of at least about 0.0001 cm², 0.0002 cm², 0.0003 cm², 0.0004 cm², 0.0005 cm², 0.0006 cm², 0.0007 cm², 0.0008 cm², 0.0009 cm², 0.001 cm², 0.002 cm², 0.003 cm², 0.004 cm², 0.005 cm², 0.006 cm², 0.007 cm², 0.008 cm², 0.009 cm², 0.01 cm², 0.02 cm², 0.03 cm², 0.04 cm², 0.05 cm², 0.06 cm², 0.07 cm², 0.08 cm², 0.09 cm², 0.1 cm², 0.2 cm², 0.3 cm², 0.4 cm², 0.5 cm², 0.6 cm², 0.7 cm², 0.8 cm², 0.9 cm², 1 cm², 2 cm², 3 cm², 4 cm², 5 cm², 6 cm², 7 cm², 8 cm², 9 cm², 10 cm², 20 cm², 30 cm², 40 cm², 50 cm², 60 cm², 70 cm², 80 cm², 90 cm², 100 cm², 200 cm², 300 cm², 400 cm², 500 cm², 600 cm², 700 cm², 800 cm², 900 cm², 1000 cm², 2000 cm², 3000 cm², 4000 cm², 5000 cm², 6000 cm², 7000 cm², 8000 cm², 9000 cm², 10000 cm², or any values therebetween. In some embodiments, the first surface comprises a working area of at most about 10000 cm², 9000 cm², 8000 cm², 7000 cm², 6000 cm², 5000 cm², 4000 cm², 3000 cm², 2000 cm², 1000 cm², 900 cm², 800 cm², 700 cm², 600 cm², 500 cm², 400 cm², 300 cm², 200 cm², 100 cm², 90 cm², 80 cm², 70 cm², 60 cm², 50 cm², 40 cm², 30 cm², 20 cm², 10 cm², 9 cm², 8 cm², 7 cm², 6 cm², 5 cm², 4 cm², 3 cm², 2 cm², 1 cm², 0.9 cm², 0.8 cm², 0.7 cm², 0.6 cm², 0.5 cm², 0.4 cm², 0.3 cm², 0.2 cm², 0.1 cm², 0.09 cm², 0.08 cm², 0.07 cm², 0.06 cm², 0.05 cm², 0.04 cm², 0.03 cm², 0.02 cm², 0.01 cm², 0.009 cm², 0.008 cm², 0.007 cm², 0.006 cm², 0.005 cm², 0.004 cm², 0.003 cm², 0.002 cm², 0.001 cm², 0.0009 cm², 0.0008 cm², 0.0007 cm², 0.0006 cm², 0.0005 cm², 0.0004 cm², 0.0003 cm², 0.0002 cm², 0.0001 cm², or any values therebetween.

In some embodiments, the apparatus described herein may be used to manipulate a fluid. In some embodiments, the manipulated fluid comprises inorganic ions, organic ions, proteins, DNA, RNA, surfactants, oil droplets, magnetic beads, nanoparticles, microparticles, polymers, organic compounds, hormones, or combinations thereof. In some embodiments, the manipulated fluid comprises water, ethanol, isopropanol, methanol, acetone, formaldehyde, methyl ethyl ketone, acetamide, ethylene glycol, propylene glycol, dimethyl sulfoxide, dimethylformamide, acetic acid, glycerol, or combinations thereof.

### Methods for Applying Surface Coatings

In another aspect, the present disclosure provides a method of coating a surface for fluid manipulation. In some embodiments, the method includes applying a film layer to said surface and applying a liquid layer to said film layer.

In some embodiments, the surface coating may be applied by at least one of spin coating, spray coating, dip coating, needle dispensing, vapor deposition, or combinations thereof. In some embodiments, applying the film layer comprises stretching and bonding a thin film to the surface. The thin film may be stretched to eliminate wrinkles and to ensure additional smoothness. The thin film may be held on the electrode array by heat or thermal bonding, by applying a vacuum, by electrostatic forces, or by mechanical means.

In some embodiments, the surface coating is formed by spray-coating the film layer onto the surface. The film layer may be cured. The lubricating layer is then sprayed or dispensed onto the film layer.

In some embodiments, the surface coating is formed by attaching the film layer to a film-frame. The film-frame is then attached to the surface. The lubricating layer is then sprayed or dispensed onto the film layer.

In some embodiments, the film layer is formed by flowing a material over the surface, such as flowing a material through the inner channel formed by a tube. The lubricating layer may be applied to the film layer by flowing a liquid over the film layer.

In some embodiments, the film layer has a thickness from about 0.1 μm to about 1000 μm. In some embodiments, the film layer has a thickness of at least about 0.1 μm, 0.2 μm, 0.3 μm, 0.4μ, 0.5μ, 0.6 μm, 0.7 μm, 0.8μ, 0.9 μm, 1μ, 2μ, 3μ, 4μ, 5μ, 6μ, 7μ, 8 μm, 9 μm, 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1000 μm, or any values therebetween. In some embodiments, the film layer is at most about 1000 μm, 900 μm, 800 μm, 700 μm, 600 μm, 500 μm, 400 μm, 300 μm, 200 μm, 100 μm, 90 μm, 80 μm, 70 μm, 60 μm, 50 μm, 40 μm, 30 μm, 20 μm, 10 μm, 9 μm, 8 μm, 7 μm, 6 μm, 5 μm, 4 μm, 3 μm, 2 μm, 1 μm, 0.9 μm, 0.8 μm, 0.7 μm, 0.6 μm, 0.5 μm, 0.4 μm, 0.3 μm, 0.2 μm, 0.1 μm or any values therebetween.

In some embodiments, the film layer is a smooth surface. A fluid manipulating surface is smooth if there is at least one 10000 μm² portion of the surface which is meant to come in contact with a manipulated fluid where any 2000 μm² portion has an Ra less than 10 μm and a Wenzel roughness factor below 2. In some embodiments, the film layer has an Ra of about 100 μm to about 0 μm. In some embodiments, the film layer has an Ra of about 100 nanometers (nm) to about 0 nm. In some embodiments, the film layer has an Ra of least about 0 μm, 0.01 μm, 0.02 μm, 0.03 μm, 0.04 μm, 0.05 μm, 0.06 μm, 0.07 μm, 0.08 μm, 0.09 μm, 0.1 μm, 0.2 μm, 0.3 μm, 0.4 μm, 0.5 μm, 0.6 μm, 0.7 μm, 0.8 μm, 0.9 μm, 1 μm, 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, or any values therebetween. In some embodiments, the film layer has an Ra of at most about μm, 90 μm, 80 μm, 70μ, 60 μm, 50μ, 40 μm, 30 μm, 20 μm, 10μ, 9μ, 8μ, 7μ, 6μ, 5μ, 4μ, 3 μm, 2 μm, 1 μm, 0.9 μm, 0.8 μm, 0.7 μm, 0.6 μm, 0.5 μm, 0.4 μm, 0.3 μm, 0.2 μm, 0.1 μm, 0.09 μm, 0.08 μm, 0.07 μm, 0.06 μm, 0.05 μm, 0.04 μm, 0.03 μm, 0.02 μm, 0.01 μm, 0 μm, or any values therebetween.

In some embodiments, the film layer comprises one or more polymeric films inorganic films, composite films, or combinations thereof. In some embodiments, the film layer is a composite film layer comprising two of more films laminated together. In some embodiments, the film layer is a composite film layer comprising three of more films laminated together. In some embodiments, the film layer is a composite film layer comprising four of more films laminated together. In some embodiments, the film layer is a composite film layer comprising five of more films laminated together. There composite films take advantage of the various properties of each material used.

In some embodiments, the film layer may comprise insulating dielectric materials. In some embodiments, the film layer comprises polyethylene, polypropylene, polystyrene, polyetheretherketone (PEEK), polyimide, polyacetal, polysulfone, polyphenylene ether, polyphenylene Sulfide (PPS), polyvinyl chloride, synthetic rubber, natural rubber, neoprene, nylon, polyacrylonitrile, polyvinyl butyral, silicone, parafilm, polyethylene terephthalate, polybutylene terephthalate, polyamides, polyoxymethlyene, polycarbonate, polymethylpentene, polyphenylene oxide (Polyphenyl ether), polyphthalamide (PPA), polylactic acid, synthetic cellulose ethers (e.g., methyl cellulose, ethyl cellulose, propyl cellulose, hydroxyethyl cellulose, hydroxyethyl cellulose, hydroxypropyl cellulose (HPC), hydroxyethyl methyl cellulose, hydroxypropyl methyl cellulose (HPMC), ethyl hydroxyethyl cellulose), paraffins, microcrystalline wax, epoxy, polytetrafluoroethylene (PTFE), tetrafluoroethylene (TFE), fluorinated ethylenepropylene copolymer (FEP), polyvinylidene fluoride (PVDF), perfluoroalkoxytetrafluoroethylene copolymer (PFA), perfluoromethyl vinylether copolymer (MFA), ethylenechlorotrifluoroethylene copolymer (ECTFE), ethylene-tetrafluoroethylene copolymer (ETFE), perfluoropolyether (PFPE), polychlorotetrafluoroethylene (PCTFE), ceramic, borosilicate glass, quartz, alumina, silica, clay minerals, bentonite, kaolinite, vermiculite, graphite, molybdenum disulfide, mica, boron nitride, sodium formate, sodium oleate, sodium palmitate, sodium sulfate, sodium alginate, other polymer materials, other ceramic materials, or combinations thereof.

In some embodiments, the film layer may be modified. In some embodiments, the film layer may be modified by either applying a secondary coating to the film layer or by functionalizing the surface of the film layer. Either the secondary coating or surface functionalization may be selected to improve the affinity of the film layer for the liquid layer. Modification of the film layer may be accomplished in either the liquid phase or gas phase. The film layer may be modified on either side, and both sides of the film layer may comprise the same or different modifications. The film layer may be modified to improve hydrophobicity and fluid manipulation.

In some embodiments, the modifications may be selected to provide other properties such as durability, dielectric breakdown, electric resistivity, dielectric constant, environmental impact, elasticity, coefficient of thermal expansion, thermal conductivity, or combinations thereof.

In some embodiments, the liquid layer may diffuse into the film layer causing the film layer to swell.

In some embodiments, the liquid layer is non-uniform. In some embodiments, the liquid layer has an average initial thickness from about 0.1 μm to about 500 μm. In some embodiments, the liquid layer has a thickness of at least about 0.1 μm, 0.2 μm, 0.3 μm, 0.4 μm, 0.5 μm, 0.6 μm, 0.7 μm, 0.8 μm, 0.9 μm, 1 μm, 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 200 μm, 300 μm, 400 μm, 500 μm, or any values therebetween. In some embodiments, the liquid layer has a thickness of at most about 500 μm, 400 μm, 300 μm, 200 μm, 100 μm, 90 μm, 80 μm, 70 μm, 60 μm, 50 μm, 40 μm, 30 μm, 20 μm, 10 μm, 9 μm, 8 μm, 7 μm, 6 μm, 5 μm, 4 μm, 3 μm, 2 μm, 1 μm, 0.9 μm, 0.8 μm, 0.7 μm, 0.6 μm, 0.5 μm, 0.4 μm, 0.3 μm, 0.2 μm, 0.1 μm, or any values therebetween.

The viscosity of the liquid layer is selected to optimize fluid mobility, reduce drag, and increase durability of the liquid layer. In some embodiments, the liquid layer has a viscosity of about 0.5 cSt to about 100 cSt. In some embodiments, the liquid layer has a viscosity of about 0 cSt to about 20 cSt. In some embodiments, the liquid layer has a viscosity of about 5 cSt to about 20 cSt. In some embodiments, the liquid layer has a viscosity of at least about 0 cSt, 0.1 cSt, 0.2 cSt, 0.3 cSt, 0.4 cSt, 0.5 cSt, 0.6 cSt, 0.7 cSt, 0.8 cSt, 0.9 cSt, 1 cSt, 2 cSt, 3 cSt, 4 cSt, 5 cSt, 6 cSt, 7 cSt, 8 cSt, 9 cSt, 10 cSt, 20 cSt, 30 cSt, 40 cSt, 50 cSt, 60 cSt, 70 cSt, 80 cSt, 90 cSt, 100 cSt, or any values therebetween. In some embodiments, the liquid layer has a viscosity of at most about 100 cSt, 90 cSt, 80 cSt, 70 cSt, 60 cSt, 50 cSt, 40 cSt, 30 cSt, 20 cSt, 10 cSt, 9 cSt, 8 cSt, 7 cSt, 6 cSt, 5 cSt, 4 cSt, 3 cSt, 2 cSt, 1 cSt, 0.9 cSt, 0.8 cSt, 0.7 cSt, 0.6 cSt, 0.5 cSt, or any values therebetween. Values provided herein for viscosity of the liquid layer may be measured when the liquid layer is at room temperature.

In some embodiments, the liquid layer has a static contact angle with the film layer of about 10 degrees or less. The small static contact angle helps to improve lubricity and reduces fouling and pinning during fluid manipulation. In some embodiments, the liquid layer has a static contact angle with the film layer of at least about 0.1 degrees, 0.2 degrees, 0.3 degrees, 0.4 degrees, 0.5 degrees, 0.6 degrees, 0.7 degrees, 0.8 degrees, 0.9 degrees, 1 degree, 2 degrees, 3 degrees, 4 degrees, 5 degrees, 6 degrees, 7 degrees, 8 degrees, 9 degrees, 10 degrees, or any values therebetween. In some embodiments, the liquid layer has a static contact angle with the film layer of at most about 10 degrees, 9 degrees, 8 degrees, 7 degrees, 6 degrees, 5 degrees, 4 degrees, 3 degrees, 2 degrees, 1 degree, 0.9 degrees, 0.8 degrees, 0.7 degrees, 0.6 degrees, 0.5 degrees, 0.4 degrees, 0.3 degrees, 0.2 degrees, 0.1 degrees, or any values therebetween.

In some embodiments the liquid layer is a lubricating layer. The lubricating layer improves overall fluid mobility by reducing friction between the film layer and the droplet, preventing droplet pinning, reducing fouling, and reducing contact angle hysteresis. Good fluid mobility is defined by the ability to move a fluid accurately, reliably, at a high speed, for extended periods of time, without pinning and fouling on the surface.

In some embodiments, the lubricating layer is selected for its affinity for the film layer and its immiscibility with the manipulated fluid. In some embodiments, the lubricating layer is a hydrocarbon layer, a silicone layer, a fluorinated layer, or combinations thereof.

In some embodiments, the lubricating layer comprises polydimethylsiloxanes, polymethyl hydrogen siloxane/hydrogen silicone oil, amino silicone oil, phenyl methyl silicone oil, diphenyl silicone oil, vinyl silicone oil, hydroxy silicone oil, cyclosiloxanes, polyalkylene oxide silicones, silicone resins, perfluoropolyether (PFPE), perfluoroalkanes, fluorinated ionic fluid, fluorinated silicone oils, perfluoroalkylether, perfluoro tri-n-butylamine (FC-40), hydrofluoroether (HFE) liquids, ionic liquids, mineral oils, ferrofluids, polyphenyl ether, vegetable oil, esters of saturated fatty and dibasic acids, grease, fatty acids, triglycerides, polyalphaolefin, polyglycol hydrocarbons, other alkanes, other non-hydrocarbon synthetic oils, or combinations thereof.

In some embodiments, the lubricating layer may comprise an additive. In some embodiments, the additive is a rheology modifier, filler, solvent, surfactant, dye, or combinations thereof. Rheology modifiers, fillers, and solvents, Rheology modifiers, fillers and solvents may help tune the viscosity of the liquid and can give non-Newtonian flow properties to the liquid. Fillers may help improve material properties such as thermal conductivity of dielectric constant and may also change rheological properties. Surfactants and solvents can help tune the surface energy of the lubricating layer with air and the manipulated fluid.

In some embodiments, a part of, or all of the surface coating may be removed and replaced. In some embodiments, the surface coating may be used once. In some embodiments, the surface coating may be used multiple times. In some embodiments, the coating may be permanent.

In some embodiments, the surface coatings described herein may be applied to a surface intended to contact a fluid. In some embodiments, non-limiting examples of a surface intended to contact a fluid may include a cannula, connector, catheter (e.g., central line, peripherally inserted central catheter (PICC) line, urinary, vascular, peritoneal dialysis, and central venous catheters), catheter connector (e.g., Leur-Lok and needleless connectors), clamp, skin hook, cuff, retractor, shunt, needle, capillary tube, endotracheal tube, ventilator, associated ventilator tubing, drug delivery vehicle, syringe, microscope slide, plate, film, laboratory work surface, well, well plate, Petri dish, tile, jar, flask, beaker, vial, test tube, tubing connector, column, container, cuvette, bottle, drum, vat, tank, organ, organ implant, or organ component (e.g., intrauterine device, defibrillator, corneal, breast, knee replacement, and hip replacement implants), artificial organ or a component thereof (e.g., heart valve, ventricular assist devices, total artificial hearts, cochlear implant, visual prosthetic, and components thereof), dental tool, dental implant (e.g., root form, plate form, and subperiosteal implants), biosensor (e.g., glucose and insulin monitor, blood oxygen sensor, hemoglobin sensor, biological microelectromechanical devices (bioMEMs), sepsis diagnostic sensor, and other protein and enzyme sensors), bioelectrode, endoscope (hysteroscope, cystoscope, amnioscope, laparoscope, gastroscope, mediaslinoscope, bronchoscope, esophagoscope, rhinoscope, arthroscope, proctoscope, colonoscope, nephroscope, angioscope, thoracoscope, esophagoscope, laryngoscope, and encepbaloscope) wound dressing (e.g., bandages, sutures, staples), and combinations thereof.

In some embodiments, the coatings described herein may be applied to a device, such as research and diagnostic arrays. Examples of research and diagnostic arrays may include sample preparation, amplification, rolling circle amplification, bridge amplification, sequencing, circular consensus sequencing, next generation sequencing, polymerase chain reaction, enzymatic polymer synthesis, and sample detection arrays.

### EXAMPLES

The following examples are included for illustrative purposes only and are not intended to limit the scope of the invention.

### Example 1: Preparation of a Replaceable Surface Coating for an Electrowetting

A replaceable surface coating was prepared for electrowetting. A substrate having an array of electrodes was coated with a parylene sealant layer. The thickness of the parylene sealant layer was between 0.1 μm and 50 μm, such as, for example, 2 μm. A silicone oil gap-filling liquid was then applied to the parylene coated substrate. The silicone oil gap-filling liquid had a viscosity between 0.65 cSt and 1,000 cSt, such as, for example, 5 cSt. A silicone coated PET thin-film was then applied over the gap-filling liquid. Alternatively, the PET thin film may be applied prior to applying the silicone gap-filling liquid. The thickness of the PET thin film was between 0.5 μm and 1000 μm, such as, for example, 13 μm. A silicone oil liquid layer, having a thickness between 50 nm and 500 μm, was then applied on top of the PET thin film. The silicone oil liquid layer had a viscosity between 0.65 cSt and 1,000 cSt, such as, for example, 5 cSt. The coating may be removed leaving behind the substrate, electrodes, and part of the sealant layer.

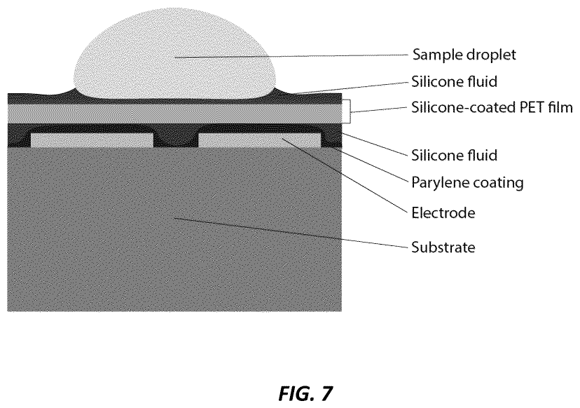

FIG. 7 is an illustration depicting the construction of a replaceable surface coating for electrowetting.

### Example 2: Preparation of a Permanent Surface Coating for an Electrowetting

A permanent surface coating was prepared for electrowetting. A substrate having an array of electrodes was coated with a thin parylene sealant layer. The thickness of the parylene sealant layer was between 0.1 μm and 50 μm, such as, for example, 2 μm. A silicone coating is applied over the parylene sealant layer. The thickness of the silicone coating was between 50 nm and 25 μm, such as, for example, 1 μm. A silicone oil liquid layer was then applied over the silicone coating. The silicone oil liquid layer had a viscosity between 0.65 cSt and 1,000 cSt, such as, for example, 5 cSt.

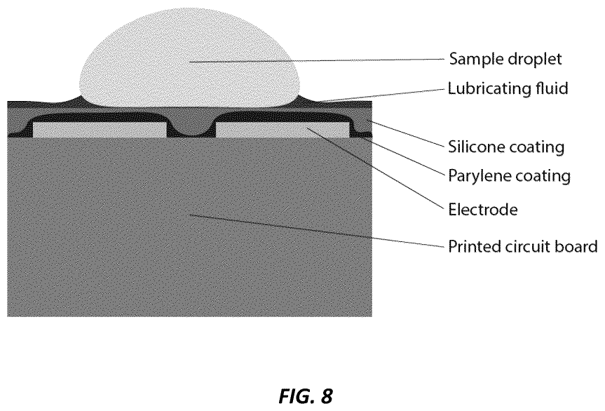

FIG. 8 is an illustration depicting the construction of a permanent surface coating for electrowetting.

### Example 3: Preparation of a Replaceable Surface Coating for Gravitational Fluid Manipulation

A replaceable surface coating was prepared for gravitational fluid manipulation. A substrate was coated with a silicone oil gap-filling liquid. The silicone oil gap-filling liquid had a viscosity between 0.65 cSt and 1,000 cSt, such as, for example, 5 cSt. A silicone coated PET thin film layer was then applied over the gap-filling liquid. The thickness of the PET thin film layer was between 0.5 μm and 1000 μm, such as, for example, 13 μm. A silicone oil liquid layer, having a thickness between 50 nm and 25 μm, such as, for example 1 μm, was then applied over the PET thin film. The coating may be removed leaving behind the substrate and part of the gap-filling liquid.

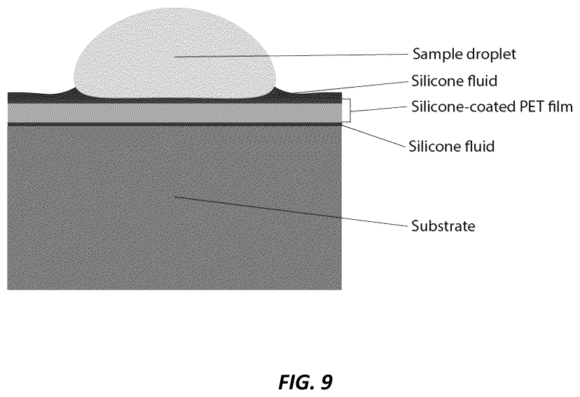

FIG. 9 is an illustration depicting the construction of a replaceable surface coating for gravitational fluid manipulation.

### Example 4: Coating of a Surface for Contacting a Fluid

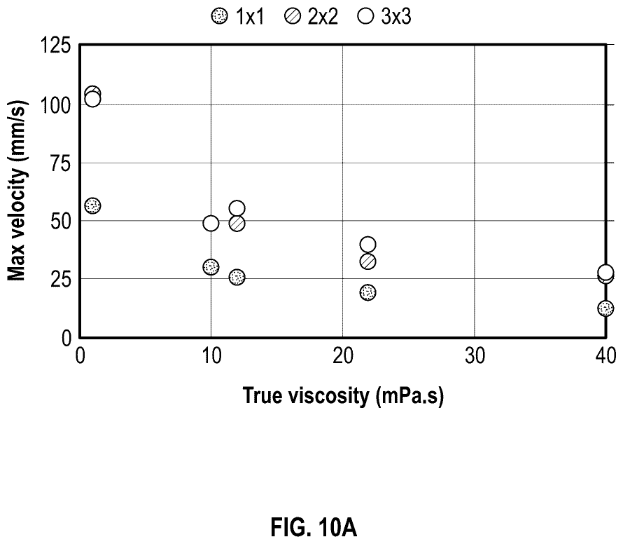

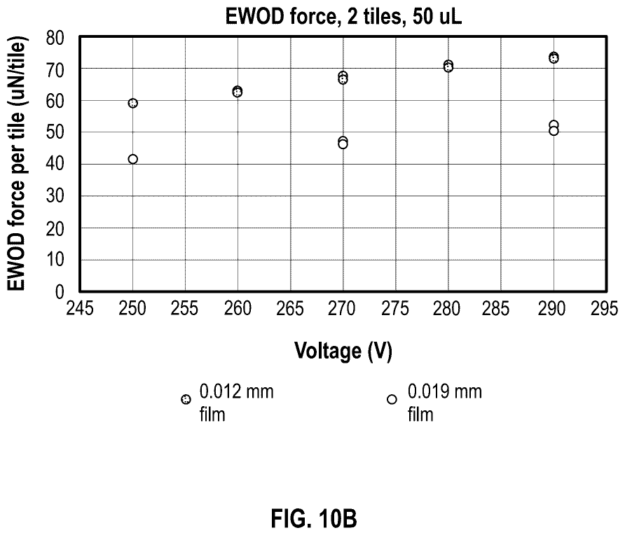

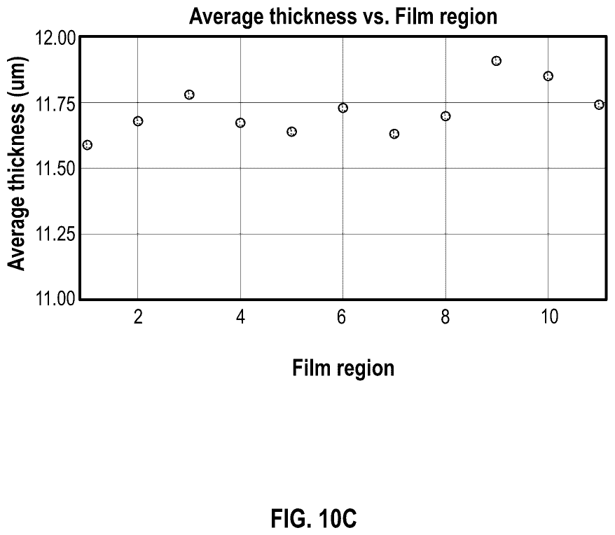

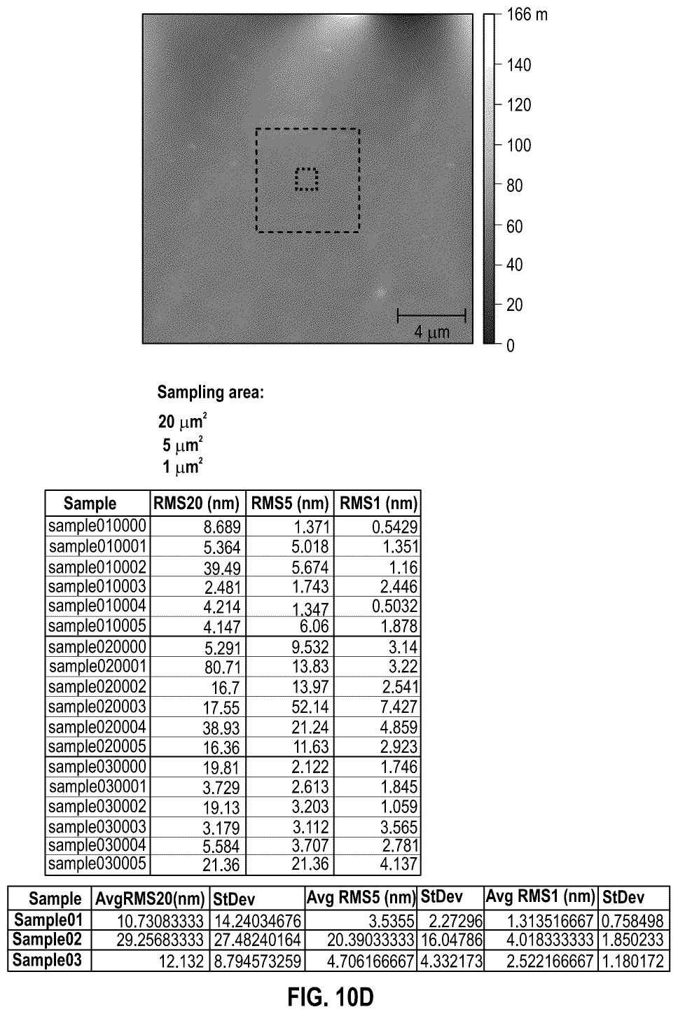

A thin film layer was applied to a surface. The thin film layer was non-textured (e.g., completely smooth, substantially smooth, smooth to within an acceptable or predetermined threshold, etc.). A liquid layer was applied to the film layer. The liquid layer was an oil layer. FIG. 10A provides example data depicting maximum droplet velocity as a function of the true viscosity of the liquid layer. In general, as the true viscosity of the liquid layer increased, the maximum droplet velocity decreased. Note, 1 mPa*s=1 cSt. The liquid layer has a static contact angle with the thin film layer of less than 10 degrees. FIG. 10B provides example data depicting electrowetting on dielectric (EWOD) force measured at various electrowetting voltage. The EWOD force was measured via a sliding angle measurement of two different thin film layers (one of 0.012 mm in thickness and the other at 0.019 mm in thickness) at various electrowetting voltages between 250 volts and 290 volts. The thicker thin film layer consistently measured lower electrowetting forces than the thinner thin film layer. FIG. 10C provides example data depicting thickness data for various film regions of the thin film layer. As depicted, the thickness varies between about 11.5 μm and about 12.0 μm. FIG. 10D provides example data depicting roughness values for three different thin film layers with different sampling area sizes of 20 μm², 5 μm², and 1 μm². As depicted, the Ra roughness for sample 1 has an average RMS20 (corresponding to the sampling area of 20 μm²) of about 10.73 nm, an average RMS5 (corresponding to the sampling area of 5 μm²) of about 3.54 nm, and an average RMS1 (corresponding to the sampling area of 1 μm²) of about 1.31 nm. As depicted, the Ra roughness for sample 2 has an average RMS20 of about 29.26 nm, an average RMS5 of about 20.39 nm, and an average RMS1 of about 4.02 nm. As depicted, the Ra roughness for sample 3 has an average RMS20 of about 12.13 nm, an average RMS5 of about 4.71 nm, and an average RMS1 of about 2.52 nm.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

What is claimed is:

1. A method of coating a surface for contacting a fluid comprising:
applying a film layer to said surface, wherein said film layer is non-textured;
applying a liquid layer to said film layer, wherein said liquid layer has a viscosity of about 0.5 centistokes (cSt) to about 100 cSt and an average initial thickness ranging from about 0.01 micrometers to about 500 micrometers.

2. The method of claim 1, wherein the film layer has a Ra of about 100 μm to about 0 μm.

3. The method of claim 1 or 2, wherein said liquid layer is a lubricating layer.

4. The method of claim 3, wherein said lubricating layer is a hydrocarbon layer, a silicone layer, a fluorinated layer, or combinations thereof.

5. The method of claim 3, wherein said lubricating layer comprises polydimethylsiloxanes, polymethyl hydrogen siloxane/hydrogen silicone oil, amino silicone oil, phenyl methyl silicone oil, Diphenyl silicone oil, vinyl silicone oil, hydroxy silicone oil, cyclosiloxanes, polyalkylene oxide silicones, silicone resins, perfluoropolyether (PFPE), perfluoroalkanes, fluorinated ionic fluid, fluorinated silicone oils, perfluoroalkylether, perfluoro tri-n-butylamine (FC-40), hydrofluoroether (HFE) liquids, ionic liquids, mineral oils, ferrofluids, polyphenyl ether, vegetable oil, esters of saturated fatty and dibasic acids, grease, fatty acids, triglycerides, polyalphaolefin, polyglycol hydrocarbons, other alkanes, or other non-hydrocarbon synthetic oils.

6. The method of any one of claims 1 to 5, wherein said liquid layer further comprises at least one additive.

7. The method of claim 6, wherein said at least one additive is a rheology modifier, filler, solvent, surfactant, dye, or combinations thereof.

8. The method of any one of claims 1 to 7, wherein said liquid layer may diffuse into said film layer causing said film layer to swell.

9. The method of any one of claims 1 to 8, wherein said liquid layer has a static contact angle with said film layer of about 10 degrees to about 0 degrees.

10. The method of any one of claims 1 to 9, wherein said liquid layer has a static contact angle with said film layer of about 5 degrees to about 0 degrees.

11. The method of any one of claims 1 to 10, wherein said film layer comprises one or more polymeric films, inorganic films, composite films, or combinations thereof.

12. The method of any one of claims 1 to 11, wherein said film layer comprises polyethylene, polypropylene, polystyrene, polyetheretherketone (PEEK), polyimide, polyacetal, polysulfone, polyphenylene ether, polyphenylene Sulfide (PPS), polyvinyl chloride, synthetic rubber, natural rubber, neoprene, nylon, polyacrylonitrile, polyvinyl butyral, silicone, parafilm, polyethylene terephthalate, polybutylene terephthalate, polyamides, polyoxymethlyene, polycarbonate, polymethylpentene, polyphenylene oxide (Polyphenyl ether), polyphthalamide (PPA), polylactic acid, synthetic cellulose ethers (e.g., methyl cellulose, ethyl cellulose, propyl cellulose, hydroxyethyl cellulose, hydroxyethyl cellulose, hydroxypropyl cellulose (HPC), hydroxyethyl methyl cellulose, hydroxypropyl methyl cellulose (HPMC), ethyl hydroxyethyl cellulose), paraffins, microcrystalline wax, epoxy, polytetrafluoroethylene (PTFE), tetrafluoroethylene (TFE), fluorinated ethylenepropylene copolymer (FEP), polyvinylidene fluoride (PVDF), perfluoroalkoxytetrafluoroethylene copolymer (PFA), perfluoromethyl vinylether copolymer (MFA), ethylenechlorotrifluoroethylene copolymer (ECTFE), ethylene-tetrafluoroethylene copolymer (ETFE), perfluoropolyether (PFPE), polychlorotetrafluoroethylene (PCTFE), ceramic, borosilicate glass, quartz, alumina, silica, clay minerals, bentonite, kaolinite, vermiculite, graphite, molybdenum disulfide, mica, boron nitride, sodium formate, sodium oleate, sodium palmitate, sodium sulfate, sodium alginate, or any other polymer or ceramic material, or combinations thereof.

13. The method of claim 1 or 12, further comprising modifying the film layer.

14. The method of claim 13, wherein modifying comprises either surface functionalization or application of a secondary coating.

15. The method of claim 13 or 14, wherein said modifying increases the affinity of said liquid layer for said film layer.

16. The method of any one of claims 1 to 15, wherein said film layer has a thickness from about 0.1 μm to about 1000 μm.

17. The method of any one of claims 1 to 16, wherein a part of, or all of said surface coating may be removed or replaced.

18. The method of any one of claims 1 to 16, wherein said surface coating may be permanent.

19. The method of any one of claims 1 to 18, wherein said surface coating is applied to a surface intended to contact a fluid.

20. The method of any one of claims 1 to 19, wherein said surface coating is applied to a surface of a cannula, connector, catheter (e.g., central line, peripherally inserted central catheter (PICC) line, urinary, vascular, peritoneal dialysis, and central venous catheters), catheter connector (e.g., Leur-Lok and needleless connectors), clamp, skin hook, cuff, retractor, shunt, needle, capillary tube, endotracheal tube, ventilator, associated ventilator tubing, drug delivery vehicle, syringe, microscope slide, plate, film, laboratory work surface, well, well plate, Petri dish, tile, jar, flask, beaker, vial, test tube, tubing connector, column, container, cuvette, bottle, drum, vat, tank, organ, organ implant, or organ component (e.g., intrauterine device, defibrillator, corneal, breast, knee replacement, and hip replacement implants), artificial organ or a component thereof (e.g., heart valve, ventricular assist devices, total artificial hearts, cochlear implant, visual prosthetic, and components thereof), dental tool, dental implant (e.g., root form, plate form, and subperiosteal implants), biosensor (e.g., glucose and insulin monitor, blood oxygen sensor, hemoglobin sensor, biological microelectromechanical devices (bioMEMs), sepsis diagnostic sensor, and other protein and enzyme sensors), bioelectrode, endoscope (hysteroscope, cystoscope, amnioscope, laparoscope, gastroscope, mediaslinoscope, bronchoscope, esophagoscope, rhinoscope, arthroscope, proctoscope, colonoscope, nephroscope, angioscope, thoracoscope, esophagoscope, laryngoscope, and encepbaloscope) wound dressing (e.g., bandages, sutures, staples), and combinations thereof.

21. A surface coating for contacting a fluid, comprising:
a film layer, wherein said film layer is non-textured; and
a liquid layer, wherein said liquid layer has a viscosity of about 0.5 cSt to about 100 cSt and an average initial thickness ranging from about 0.01 μm to about 500 μm.

22. The coating of claim 21, wherein the film layer has a Ra of about 100 μm to about 0 μm.

23. The coating of claim 21 or 22, wherein said liquid layer is a lubricating layer.

24. The coating of claim 23, wherein said lubricating layer is a hydrocarbon layer, a silicone layer, a fluorinated layer, or combinations thereof.

25. The coating of claim 23, wherein said lubricating layer comprises polydimethylsiloxanes, polymethyl hydrogen siloxane/hydrogen silicone oil, amino silicone oil, phenyl methyl silicone oil, Diphenyl silicone oil, vinyl silicone oil, hydroxy silicone oil, cyclosiloxanes, polyalkylene oxide silicones, silicone resins, perfluoropolyether (PFPE), perfluoroalkanes, fluorinated ionic fluid, fluorinated silicone oils, perfluoroalkylether, perfluoro tri-n-butylamine (FC-40), hydrofluoroether (HFE) liquids, ionic liquids, mineral oils, ferrofluids, polyphenyl ether, vegetable oil, esters of saturated fatty and dibasic acids, grease, fatty acids, triglycerides, polyalphaolefin, polyglycol hydrocarbons, other alkanes, or other non-hydrocarbon synthetic oils.

26. The coating of any one of claims 23 to 25, wherein said lubricating layer further comprises at least one additive.

27. The coating of claim 26, wherein said at least one additive is a rheology modifier, filler, solvent, surfactant, dye, or combinations thereof.

28. The coating of any one of claims 21 to 27, wherein said liquid layer may diffuse into said film layer causing said film layer to swell.

29. The coating of any one of claims 21 to 28, wherein said liquid layer has a static contact angle with said film layer of about 10 degrees or less.

30. The coating of any one of claims 21 to 29, wherein said liquid layer has a static contact angle with said film layer of about 5 degrees or less.

31. The coating of any one of claims 21 to 30, wherein said film layer comprises one or more polymeric films inorganic films, composite films, or combinations thereof.

32. The coating of any one of claims 21 to 31, wherein said film layer comprises polyethylene, polypropylene, polystyrene, polyetheretherketone (PEEK), polyimide, polyacetal, polysulfone, polyphenylene ether, polyphenylene Sulfide (PPS), polyvinyl chloride, synthetic rubber, natural rubber, neoprene, nylon, polyacrylonitrile, polyvinyl butyral, silicone, parafilm, polyethylene terephthalate, polybutylene terephthalate, polyamides, polyoxymethlyene, polycarbonate, polymethylpentene, polyphenylene oxide (Polyphenyl ether), polyphthalamide (PPA), polylactic acid, synthetic cellulose ethers (e.g., methyl cellulose, ethyl cellulose, propyl cellulose, hydroxyethyl cellulose, hydroxyethyl cellulose, hydroxypropyl cellulose (HPC), hydroxyethyl methyl cellulose, hydroxypropyl methyl cellulose (HPMC), ethyl hydroxyethyl cellulose), paraffins, microcrystalline wax, epoxy, polytetrafluoroethylene (PTFE), tetrafluoroethylene (TFE), fluorinated ethylenepropylene copolymer (FEP), polyvinylidene fluoride (PVDF), perfluoroalkoxytetrafluoroethylene copolymer (PFA), perfluoromethyl vinylether copolymer (MFA), ethylenechlorotrifluoroethylene copolymer (ECTFE), ethylene-tetrafluoroethylene copolymer (ETFE), perfluoropolyether (PFPE), polychlorotetrafluoroethylene (PCTFE), ceramic, borosilicate glass, quartz, alumina, silica, clay minerals, bentonite, kaolinite, vermiculite, graphite, molybdenum disulfide, mica, boron nitride, sodium formate, sodium oleate, sodium palmitate, sodium sulfate, sodium alginate, or any other polymer or ceramic material.

33. The coating of any one of claims 21 to 32, wherein said film layer is modified.

34. The coating of claim 33, wherein said modified film layer comprises either a functionalized surface or a secondary coating.

35. The coating of claim 33 or 34, wherein said modified film layer has a higher affinity for said liquid layer.

36. The coating of any one of claims 21 to 35, wherein said film layer has a thickness from about 0.1 μm to 1000 μm.

37. The coating of any one of claims 21 to 36, wherein a part of, or all of said coating may be removed or replaced.

38. The coating of any one of claims 21 to 36, wherein said coating may be permanent.

39. The coating of any one of claims 21 to 38, wherein said coating is applied to a surface intended to contact a fluid.

40. The coating of any one of claims 21 to 39, wherein said coating is applied to a surface of a cannula, connector, catheter (e.g., central line, peripherally inserted central catheter (PICC) line, urinary, vascular, peritoneal dialysis, and central venous catheters), catheter connector (e.g., Leur-Lok and needleless connectors), clamp, skin hook, cuff, retractor, shunt, needle, capillary tube, endotracheal tube, ventilator, associated ventilator tubing, drug delivery vehicle, syringe, microscope slide, plate, film, laboratory work surface, well, well plate, Petri dish, tile, jar, flask, beaker, vial, test tube, tubing connector, column, container, cuvette, bottle, drum, vat, tank, organ, organ implant, or organ component (e.g., intrauterine device, defibrillator, corneal, breast, knee replacement, and hip replacement implants), artificial organ or a component thereof (e.g., heart valve, ventricular assist devices, total artificial hearts, cochlear implant, visual prosthetic, and components thereof), dental tool, dental implant (e.g., root form, plate form, and subperiosteal implants), biosensor (e.g., glucose and insulin monitor, blood oxygen sensor, hemoglobin sensor, biological microelectromechanical devices (bioMEMs), sepsis diagnostic sensor, and other protein and enzyme sensors), bioelectrode, endoscope (hysteroscope, cystoscope, amnioscope, laparoscope, gastroscope, mediaslinoscope, bronchoscope, esophagoscope, rhinoscope, arthroscope, proctoscope, colonoscope, nephroscope, angioscope, thoracoscope, esophagoscope, laryngoscope, and encepbaloscope) wound dressing (e.g., bandages, sutures, staples), and combinations thereof.

41. An apparatus for fluid manipulation, comprising:
a substrate comprising one or more electrodes; and
a first surface over said substrate, wherein said first surface comprises the coating of any one of claims 21 to 40.

42. The apparatus of claim 41, wherein said substrate further comprises a sealant layer.

43. The apparatus of claim 42, wherein said sealant layer comprises fluoropolymers, polyurethanes, acrylics, silicones, polyolefins, parylenes, or combinations thereof.

44. The apparatus of claim 41 or 42, wherein said first surface is flat, curved, tubular, horizontal, vertical, or any combinations thereof.

45. The apparatus of any one of claims 41 to 44, further comprising a second surface parallel to the first surface.

46. The apparatus of any one of claims 41 to 45, further comprising a gap-filling liquid between said substrate and said first surface.

47. The apparatus of claim 46, wherein said gap-filling liquid forms displaced volume between said substrate and said first surface.

48. The apparatus of claim 47, wherein said displaced volume has a height between about 0.01 μm to about 500 μm.

49. The apparatus of any one of claims 46 to 48, wherein said gap-filling liquid is a gel, paste, grease, high viscosity oil, a low viscosity oil, or combinations.

50. The apparatus of claim 49, wherein said gap-filling liquid is a silicone paste, lithium grease, silicone grease, thermal paste, or dyed grease.

51. The apparatus of any one of claims 46 to 50, wherein said gap-filling liquid is a capillary liquid.

52. The apparatus of claim 51, wherein said capillary liquid has a contact angle with said film layer of about 5 degrees to about 0 degrees.

53. The apparatus of claim 52, wherein said capillary liquid has a contact angle with said film layer of about 1 degree to about 0 degrees.

54. The apparatus of claim 51, wherein said capillary liquid has a contact angle with said substrate or said sealant layer of about 5 degrees to about 0 degrees.

55. The apparatus of claim 54, wherein said capillary liquid has a contact angle with said substrate or said sealant layer of about 1 degree to about 0 degrees.

56. The apparatus of any one of claims 51 to 55, wherein said capillary liquid is a hydrocarbon oil, silicone oil, fluorinated oil, or liquid acrylate.

57. The apparatus of any one of claims 51 to 56, wherein said gap-filling liquid further comprises at least one additive.

58. The apparatus of claim 57, wherein said at least one additive is a rheology modifier, filler, solvent, surfactant, dye, or combinations thereof.

59. The apparatus of any one of claims 51 to 58, wherein said gap-filling liquid is either an insulating or conductive liquid.

60. The apparatus of any one of claims 46 to 58, wherein said first surface is modified.

61. The apparatus of claim 60, wherein said modified first surface comprises either a functionalized surface or a secondary coating.

62. The apparatus of claim 60 or 61, wherein said modified first surface has a higher affinity for said gap-filling liquid.

63. The apparatus of any one of claims 41 to 45, further comprising a vacuum between said substrate and said first surface.

64. The apparatus of any one of claims 41 to 63, wherein said first surface of said apparatus has a working area of about 0.0001 cm² to about 10000 cm².

65. The apparatus of any one of claims 41 to 64, wherein said apparatus further comprises a frame configured to support said first surface.

66. The apparatus of any one of claims 41 to 65, wherein said first surface of said apparatus is configured to contact a fluid to be manipulated.

67. The apparatus of any one of claims 41 to 66, wherein said apparatus is configured to manipulate said fluid in contact with said first surface.

68. The apparatus of any one of claims 41 to 67, wherein the manipulated fluid comprises inorganic ions, organic ions, proteins, DNA, RNA, surfactants, oil droplets, magnetic beads, nanoparticles, microparticles, polymers, organic compounds, hormones, or combinations thereof.

69. The apparatus of any one of claims 41 to 68, wherein the manipulated fluid comprises water, ethanol, isopropanol, methanol, acetone, formaldehyde, methyl ethyl ketone, acetamide, ethylene glycol, propylene glycol, dimethyl sulfoxide, dimethylformamide, acetic acid, glycerol, or combinations thereof.

70. The method of claim 2, wherein the film layer has a Ra of about 100 nanometers (nm) to about 0 nm.

71. The coating of claim 22, wherein the film layer has a Ra of about 100 nanometers (nm) to about 0 nm.

72. The method of claim 1, wherein said viscosity of said liquid layer is between about 0.5 cSt to about 20 cSt.

73. The method of claim 72, wherein said viscosity of said liquid layer is between about 5 cSt to about 20 cSt.

74. The coating of claim 21, wherein said viscosity of said liquid layer is between about 0.5 cSt to about 20 cSt.

75. The coating of claim 74, wherein said viscosity of said liquid layer is between about 5 cSt to about 20 cSt.
